(19) United States
(12) Patent Application Publication
(10) Pub. No.: US 2020/0258603 A1
(43) Pub. Date: Aug. 13, 2020

(54) Systems and Methods for Creating and Utilizing Adaptive Care Systems

(71) Applicants: Yuri Teschler, Austin, TX (US); Jeanne Teschler, Austin, TX (US); Cody Griffin, Austin, TX (US); Dan Bitman, Austin, TX (US); Jennifer Chaney, Richmond, TX (US); William Archibald, Austin, TX (US)

(72) Inventors: Yuri Teschler, Austin, TX (US); Jeanne Teschler, Austin, TX (US); Cody Griffin, Austin, TX (US); Dan Bitman, Austin, TX (US); Jennifer Chaney, Richmond, TX (US); William Archibald, Austin, TX (US)

(21) Appl. No.: 16/271,144

(22) Filed: Feb. 8, 2019

(51) Int. Cl. G16H 10/60; G16H 50/30; G16H 20/60; G16H 20/30

(52) U.S. Cl. CPC G16H 10/60; G16H 50/30; G06F 16/22; G16H 20/30; G16H 20/60

(57) ABSTRACT

A system, method, and computer-usable medium are disclosed for creating and modifying a care plan assigned to a patient in response to observed data from the patient. In one embodiment of the method, data corresponding to one or more types of biometric diagnosis is received from the patient and a care plan is prescribed for the patient. The care plan includes one or more biometric thresholds corresponding to the one or more types of biometric information and receiving one or more reported symptoms for the patient. The care plan further includes one or more conditions corresponding to the one or more reported symptoms. Upon determining that the received data satisfy at least one of the biometric thresholds and the one or more reported symptoms satisfy at least one of the one or more conditions, an event rule for modifying the care plan based on the received data and the one or more reported symptoms is identified. The care plan is then modified based on the event rule.

### BACKGROUND OF THE INVENTION

### Field of the Invention

Embodiments of the present disclosure generally relate to health care management systems. More specifically, embodiments disclosed herein relate to systems and methods for monitoring patient healthcare and providing a health care plan that can be dynamically modified in response to plan adherence data obtained from a patient.

### Description of the Related Art

Physicians and other healthcare professionals often provide patients with care plans for treating chronic illnesses, such as diabetes. These care plans are usually a written document that provides directions and routines for the patient to follow to manage their health conditions. For example, a care plan for diabetes may include dietary restrictions, a set of tasks for the patient to perform (e.g., exercise for a given duration), written content that educates the patient about their diagnosed condition (e.g., brochures describing the diagnosed condition), and logs for the patient to record information on a scheduled basis (e.g., weight, blood pressure, blood sugar levels, etc.). As part of the treatment of the condition, the patient is expected to adhere to the tasks listed in the care plan and to then follow-up with the care provider in subsequent appointments to assess the patient's progress.

The current care plan approach has several shortcomings. For instance, a care plan for a particular condition is often tailored towards the “generalized” condition itself, without considering relevant details about a patient. Typically, once a doctor has diagnosed a patient with a particular condition, the doctor may provide a “standardized” care plan for the individual that instructs the individual on how to manage the condition. Although a standarized care plan may offer some advantages, various patients may still respond differently to the care plan. As a result, the care plan may be less effective for some than it is for others. Furthermore, the doctor often has no way of determining the patient's adherence to the care plan until a follow-up appointment. By the time the patient and doctor meet in person, the patient's memory and written records may be incomplete. The lack of complete information may, therefore, result in a suboptimal analysis of, or conclusions about, the patient's adherence to the care plan.

To address this concern, care providers currently employ call centers to contact the patient periodically and determine whether the patient is following the care plan. This approach provides some benefits to patients, but there is a clear need for a more effective and cost-efficient solution for monitoring patients with chronic conditions. The systems and methods of the present disclosure, described hereinbelow, provide such a solution.

### SUMMARY OF THE INVENTION

A system, method, and computer-usable medium are disclosed for creating and modifying a care plan assigned to a patient in response to observed data from the patient. In one embodiment of the method, data corresponding to one or more types of biometric diagnosis is received from the patient and a care plan is prescribed for the patient. The care plan includes one or more biometric thresholds corresponding to the one or more types of biometric information and receiving one or more reported symptoms for the patient. The care plan further includes one or more conditions corresponding to the one or more reported symptoms. Upon determining that the received data satisfy at least one of the biometric thresholds and the one or more reported symptoms satisfy at least one of the one or more conditions, an event rule for modifying the care plan based on the received data and the one or more reported symptoms is identified. The care plan is then modified based on the event rule.

Another embodiment includes a computer-readable storage medium for storing instructions used to create and modify the care plan. When the instructions are executed on a processor, the system performs an operation for modifying a care plan assigned to a patient in response to data observed from the patient. The care plan includes one or more biometric thresholds corresponding to the one or more types of biometric data. The processing operation includes receiving data corresponding to one or more types of biometric data for the patient. The operation also includes receiving one or more reported symptoms for the patient. The care plan includes one or more conditions corresponding to the one or more reported symptoms. Upon determining that at least one of the received data satisfies at least one of the biometric thresholds and the one or more reported symptoms satisfy at least one of the one or more conditions, an event rule is identified for modifying the care plan based on the received data and the one or more reported symptoms. The care plan is then modified based on the event rule. In some embodiments, the system sends a notification to the care team which may intervene upon receiving a notification regarding a biometric parameter or failure of the patient to adhere to one or more components of the care plan. In various embodiments of the disclosure, the care plan may include instructions to eat certain types of food in certain amounts (meal plans), instructions to take certain medications on a schedule (prescriptions) and to exercise for a predetermined length of time (exercise regimes). The system also may send notifications and reminders to encourage adherence to the care plan.

In yet another embodiment, a system comprises a processor and a memory storing one or more application programs configured to perform an operation for modifying a care plan assigned to a patient in response to data observed from the patient. The operation includes receiving data corresponding to one or more types of biometric data for the patient. The care plan includes one or more biometric thresholds corresponding to the one or more types of biometric data. The operation also includes receiving one or more reported symptoms for the patient. The care plan includes one or more conditions corresponding to the one or more reported symptoms. Upon determining that at least one of the received data satisfy at least one of the biometric thresholds and the one or more reported symptoms satisfy at least one of the one or more conditions, an event rule for modifying the care plan based on the received data and the one or more reported symptoms is identified. The care plan is modified based on the event rule.

Although the present invention discloses novel features for improvements for care plan in general, the example embodiments discussed hereinbelow relate to care plans monitoring and managing Type II diabetes. Various embodiments include systems and methods for monitoring a plurality of nodes including: blood glucose levels; adherence with scheduled medication regimens; weight maintenance; physical activity; and dietary parameters. Each of these nodes can be enabled or disabled for each patient depending on the parameters of their treatment. If a node is enabled, the patient's engagement and interaction directly determine the patient's overall adherence, as will be discussed in greater detail hereinbelow.

### BRIEF DESCRIPTION OF THE DRAWINGS

The present invention may be better understood, and its numerous objects, features and advantages made apparent to those skilled in the an by referencing the accompanying drawings. The use of the same reference number throughout the several figures designates a like or similar element.

FIG. 1 illustrates an example computing environment, according to one embodiment of the disclosure.

FIG. 2 illustrates example progressions through care plan states, according to one embodiment of the disclosure.

FIG. 3 illustrates a table listing states and associated thresholds and event rules, according to one embodiment of the disclosure.

FIG. 4 illustrates a method for modifying a care plan assigned to a patient in accordance with to one embodiment of the disclosure.

FIG. 5a is a flow chart illustration of processing steps to configure, publish, and activate a process for a care plan for the Medication Component of a Type II Diabetes care plan.

FIG. 5b is a flow chart illustration of the care plan process for patients to log adherence activity for the Medication Component of a Type II Diabetes care plan.

FIG. 5c is a flow chart illustration of the weekly care plan process for patients to log adherence activity for the Medication Component of a Type II Diabetes care plan.

FIG. 5d is a flow chart illustration of the daily care plan process for patients to log adherence activity for the Medication Component of a Type II Diabetes care plan.

FIG. 5e is a flow chart illustration of the Scheduled Medication process for monitoring patient adherence with the Medication Component of a Type II Diabetes care plan.

FIG. 5f is a flow chart illustration of the care plan process for Skipped Medication protocol the Medication Component of a Type II Diabetes care plan.

FIG. 6a is a flow chart illustration of processing steps to configure, publish, and activate a process for a care plan for the Health-Glucose component of a Type II Diabetes care plan.

FIG. 6b is a flow chart illustration of the Care Plan Process for patients to log adherence activity for the Health-Glucose component of a personal care plan.

FIG. 6c is a flow chart illustration of the weekly care plan process for patients to log adherence activity for the Health-Glucose component of a personal care plan.

FIG. 6d is a flow chart illustration of the daily care plan process for patients to log adherence activity for the Health-Glucose component of a personal care plan.

FIG. 6e is a flow chart illustration of the Scheduled Measurement component of the Health-Glucose component of a personal care plan.

FIG. 6f is a flow chart illustration of the care plan process for the Fasting Protocol for the Health-Glucose component of a personal care plan.

FIG. 6g is a flow chart illustration of the Acknowledgement protocol component of the Health-Glucose component of a patient's personal care plan.

FIG. 6h is a flow chart illustration of the Missed Scheduled Measurement component of the Health-Glucose component of a patient's personal care plan.

FIG. 6i is a flow chart illustration of the Initial Retake Protocol of the Health-Glucose component of a patient's personal care plan.

FIG. 6j is a flow chart illustration of the Low Measurement Protocol of the Health-Glucose component of a patient's personal care plan.

FIG. 6k is a flow chart illustration of the High Missed Measurement component of the Health-Glucose component of a patient's personal care plan.

FIG. 6l is a flow chart illustration of the Low Missed Measurement Protocol of the Health-Glucose component of a patient's personal care plan.

FIG. 7a is a flow chart illustration of the Configure, Publish, and Activate protocol of the Health-Weight component of a patient's personal care plan.

FIG. 7b is a flow chart illustration of the Care Plan Process protocol of the Health-Weight component of a patient's personal care plan.

FIG. 7c is a flow chart illustration of the Weekly Process protocol of the Health-Weight component of a patient's personal care plan.

FIG. 7d is a flow chart illustration of the Daily Process protocol of the Health-Weight component of a patient's personal care plan.

FIG. 7e is a flow chart illustration of the Missed Scheduled Measurement protocol of the Health-Weight component of a patient's personal care plan.

FIG. 7f is a flow chart illustration of the Exceeds Weight Variance protocol of the Health-Weight component of a patient's personal care plan.

FIG. 7g is a table showing parameters for Weight Mode vs. Measurement Result for the Confirmation protocol of a Health-Weight component of a patient's personal care plan.

FIG. 7h is a flow chart illustration of the Confirmation protocol of the Health-Weight component of a patient's personal care plan.

FIG. 8a is a flow chart illustration of the Configure, Publish, and Activate protocol of the Activity-Steps component of a patient's personal care plan.

FIG. 8b is a flow chart illustration of the Care Plan Process protocol of the Activity-Steps component of a patient's personal care plan.

FIG. 8c is a flow chart illustration of the Weekly Process protocol of the Activity-Steps component of a patient's personal care plan.

FIG. 8d is a flow chart illustration of the Daily Process protocol of the Activity-Steps component of a patient's personal care plan.

FIG. 8e is a flow chart illustration of the First Reminder protocol of the Activity-Steps component of a patient's personal care plan.

FIG. 8f is a flow chart illustration of the Second Reminder protocol of the Activity-Steps component of a patient's personal care plan.

FIG. 8g is a flow chart illustration of the Daily Goal Met protocol of the Activity-Steps component of a patient's personal care plan.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

A method, system and computer-usable medium are disclosed for creating and using a care plan assigned to a patient to provide in response to biometric data observed from the patient. The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

As described in greater detail below, the Type II Diabetes (T2D) care plan of the present disclosure is targeted at patients that are not dependent on insulin. Its purpose is to allow for both the patient and their care team to digitally track and monitor the management of their chronic condition. When necessary, it provides guidance to the patient for common events that would have previously involved physician intervention whether it was necessary or not.

The patient accesses and interacts with the care plan by means of a mobile app and wirelessly connected devices such as a scale, glucometer, and activity tracker. Using the care plan, the patient can provide metrics through connected devices and self-reporting, track and monitor progress, interact with the care team via secure messaging, and receive reminders, encouragement, and motivation inside and outside of the app.

The care team accesses and interacts with the patients in the care plan by means of a care team portal in a modern web browser on their computer or mobile device. The care team members can receive notifications for non-compliant patients, monitor, and take action based upon the patient pool metrics, monitor and take action based upon an individual patient's metrics, and interact with patients via secure messaging.

The care plan of the present disclosure includes the following nodes: 1) Scheduled Medication; 2) Blood Glucose; 3) Weight Management (Loss); 4) Physical Activity (Steps T2D); 5) Nutrition (Diet T2D); and 6) Nutrition Journal (T2D). These nodes can be enabled or disabled for each patient depending on the parameters of their treatment. If a node is enabled, the patient's interactions with it are used to calculate various metrics regarding their overall care plan.

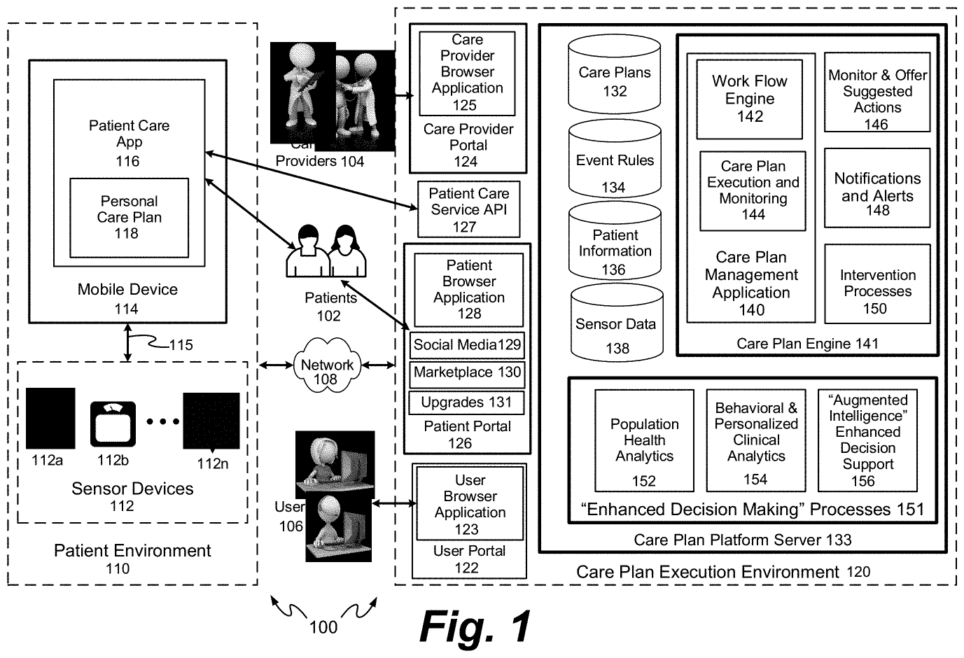

FIG. 1 illustrates an example computing environment 100 according to one embodiment of the disclosure. The computing environment 100 is operable to implement a dynamically adaptive care plan for patients 102, care providers 104, and other users 106. In the various embodiments of the disclosure, the users 106 may be third-party insurance companies, researchers, or other authorized individuals. The patient environment 110 comprises a plurality of sensors 112a-n that are operably connected to a patient 102. The sensors 112a-n may be connected to a mobile device 114 by an appropriate connection 115, which may be a physical connection such as a wire or a wireless connection, such as a Bluetooth or Wi-Fi connection. The mobile device 114 also comprises an personal care plan 118 that has been downloaded onto an app 116 on the mobile device 116 to implement a care plan for the patient 102, as will be discussed in greater detail hereinbelow.

The care plan execution environment 120 comprises a patient care service API 127 to facilitate the interaction of the respective user with the care plan execution environment 120. A patient 102 may gain access to data and services from the various processes in the care plan execution environment 120 by using the patient care app 116 that is communicatively coupled to the patient care service API 127. The patient care app 116 is also operable to transmit data from the patient sensors 112 a-n via the patient care service API 127 for storage in the sensor data storage 130.

Alternatively, patients 102 may gain access data and services from the various processes in the care plan execution environment 120 by using the patient browser application 128 of the patient portal 126. Information and services provided to users include social media services via the social media service module 129, product information via the marketplace service module 130, and software updates and upgrades via the upgrades service module 131. The user portal 122 comprises a user browser service 123; likewise, the care provider portal 124 comprises a care provider browser application 125.

The care plan platform server 133 comprises a plurality of storage modules, including care plan storage 132, event rules storage 134, patient information storage 136, and sensor data storage. The care plan storage 132 comprises a plurality of care plans that can be customized for individual patients using patient information, sensor data, and event rules, as will be discussed hereinbelow.

In various embodiments, the care plan platform server 133 is implemented to include one or more enhanced decision-making processes 151. As used herein, enhanced decision-making refers to various aspects of computer science that seek to create “intelligent” machines capable of emulating the operation of the human brain. In general, the success of these techniques is reliant upon access to objects, categories, properties, and their respective relationships.

“Machine learning” is a component of the enhanced decision-making component of the various embodiments. As used herein, machine learning refers to methods of data analysis that automate the building of analytical models that can “learn” from data, identify patterns, and make decisions without being explicitly programmed to do so. As such, machine learning can be used to learn from, and make predictions, according to various input data.

In typical implementations, machine learning may be achieved through the use of various supervised or unsupervised learning approaches. Supervised learning refers to the mapping of an input to an output according to example input-output pairs to infer a function from labeled training data. Conversely, as used herein, unsupervised learning does not rely upon feedback for training. Instead, it identifies commonalities within an initial set of data and reacts accordingly to the presence or absence of such commonalities with each new piece of data.

In various embodiments, the enhanced decision-making processes 151 may include a population health analytics 152 module, a behavioral and personalized clinical analytics 154 module, and an augmented intelligence enhanced decision support 156 module. In certain embodiments, the population health analytics 152 module is implemented to process a set of health data associated with a target population to identify certain correlations. In various embodiments, the correlations are identified through the use of various predictive analytics approaches familiar to skilled practitioners of the art. In various embodiments, the target population may be selected according to a shared ailment or disease. For example, the target population may be adults between the ages of 40 and 60 suffering from Type II diabetes. Various correlations may be made between the individual population member's age, sex, weight, activity levels at different times of the day, and various fluctuations in their respective glucose levels.

In some embodiments, the behavioral and personalized clinical analytics 154 module is implemented to perform various analytics operations by analyzing commonalities and differences between a particular patient's medical data to that of a group of similar patients. In various embodiments, the analytics operations are performed by comparing a patient's medical data to various health data provided by the population health analytics 152 module. As an example, a group of active 50 year old males in the population may participate in daily exercise sessions. Accordingly, their glucose level may be low by the time they return home from an exercise session. As a result, they need to raise their blood sugar level. In this example, analytics of fluctuations in glucose levels of a sample population of males of the same age and exercise routines may provide an indication of how much their blood sugar levels need to be raised.

In certain embodiments, the augmented intelligence enhanced decision support 156 module is implemented to provide guidance and suggestions to a healthcare provider formulating a plan of care for a patient. Certain artificial intelligence approaches, described in greater detail herein, are used to provide such guidance and suggestions. In some embodiments, various outputs resulting from the operation of the population health analytics 152 and behavioral and personalized clinical analytics 154 modules are used as inputs to the augmented intelligence enhanced decision support 156 module.

As an example, one of the individual males in the example patient population may arrive home after an exercise session with a depressed blood sugar level. Accordingly, he will eat a snack to raise his glucose level. However, the snack he chooses may have too high a sugar content, which results in a spike in his blood sugar level. In this example, the augmented intelligence enhanced decision support 156 module may be implemented to suggest taking the patient's weight into account when recommending a suitable plan of care, as the ratio between their weight and the amount of sugar being ingested may be out of balance.

The care plan engine 141 comprises a care plan management application 140 that further comprises a work flow engine 142 that has overall responsibility for managing the flow of processing tasks. The care plan execution and monitoring module 144 is operable to monitor and prioritize the work flow engine's execution of processing tasks.

The “monitor & offer suggested action module” 146 is operable to monitor the patient's biometric status based in patient information and sensor data stored in the patient information storage 136 and the sensor data 138, respectively. If a patient is out of adherence with his/her assigned care plan, the module 146 offers suggested actions to move toward adherence with the care plan. The notification and alerts module 148 monitors adherence with the suggested actions. The intervention processes module 150 provides notifications and alerts to the patients 102 based on factors discussed below.

The care protocol implemented by the care plan execution environment 120 may specify metrics that the care platform monitors during the patient's treatment, thresholds to detect for each metric, and remedial actions to be taken in response to detecting such thresholds. One example metric is blood sugar levels. The blood sugar level metric may be associated with thresholds indicating values and conditions in which the care platform performs some action in response to detecting such values and conditions, e.g., sending instructions to the patient to rest for a given period of time. Further, the care protocol may provide the patient with resources to educate the patient about a diagnosed condition, treatment, and the like (e.g., instructional videos, articles, etc.). The care platform provides a template for each care plan protocol and consolidates the configured care plan protocols into one overall care plan for the patient 102.

Once created, the care platform server 131 may transmit the personal care plan 118 to an patient care app 116 in a mobile device 114 owned by a patient 102, e.g., a smart phone or tablet device. Though the device, the patient 102 may access the care plan 118 and understand the tasks to perform. Further, the patient care plan app 118 may receive information from various sensors 112a-n that monitor patient activity (e.g., heart rate, weight, blood sugar level). The patient care app 116 also may prompt the patient 102 to report symptoms experienced while adhering to the personal care plan 118. The patient care app 118 can record the information in the personal care plan 118 and relay information to the care platform server 131. A care provider can monitor the patient's adherence to the care plan using the care provider browser application 125, as discussed above.

The care platform also may monitor the patient's adherence to the personal care plan 118 based on sensor data and reported symptoms received (e.g., from the mobile application) and, in response, automatically modify the personal care plan 118 if the observed patient biometrics and/or reported symptoms satisfy a corresponding threshold condition. If a threshold condition is satisfied, the care platform may modify the care plan based on an event rule associated with the threshold condition. Thereafter, the care platform continues to obtain sensor data (and reported symptoms) from the patient after the modification. The care platform determines whether the metrics have improved based on the changes made to the personal patient care plan.

As an example, assume that a care plan for a given patient includes a threshold for a patient's blood sugar level, e.g., specifying that over a given day, the patient's blood sugar level should not exceed X. If sensor data retrieved from the patient indicates that the blood sugar level crosses the threshold, the care platform may insert a task instructing the patient to perform some action to lower the blood sugar level, such as increase medication or perform breathing exercises for a period of time. Further, the care platform may substitute a new blood sugar level threshold, e.g., higher than the original threshold. The care platform notifies the patient of the change (i.e., through the mobile application 116) and continues to monitor the sensor data sent by the patient sensors 112a-n. As another example, the care platform could include another condition that specifies that if the patient's blood sugar level exceeds Y and the patient reports feeling the symptom of dizziness, the care platform should perform a treatment regimen aimed at reducing the patient's blood sugar level, e.g., instructing the patient to rest and perform breathing exercises. The care platform may continue to add or change tasks aimed at achieving a desired outcome.

If the care platform receives sensor data indicating that the patient's metrics are not improving, e.g., the blood sugar level has exceeded a second threshold, then the care platform may insert additional tasks and thresholds to the care plan. In addition, the care platform may alert a care provider or emergency services if the modifications to the care plan eventually prove to be ineffective.

Various features for modifying care plans will now be discussed in connection with state diagrams and flow charts. New or modified conditions may be associated with a corresponding event rule. A threshold and a corresponding event rule may reflect a given state of health of the patient 102 based on the observed biometric data and reported symptoms. Each task associated with a new state is aimed towards orienting the biometrics observed in a patient 102 towards a desired result and/or eliminating symptoms.

In one embodiment, the event rules 134 are created in accordance with patient information 136 and policies of the care provider 104. Patient information 136 may include treatment history of the patient 102, such as medications previously prescribed and whether the medications had a beneficial or adverse effect towards the patient. The care plan management application 140 may add tasks known to be effective in treating the patient 102, based on the patient information 136.

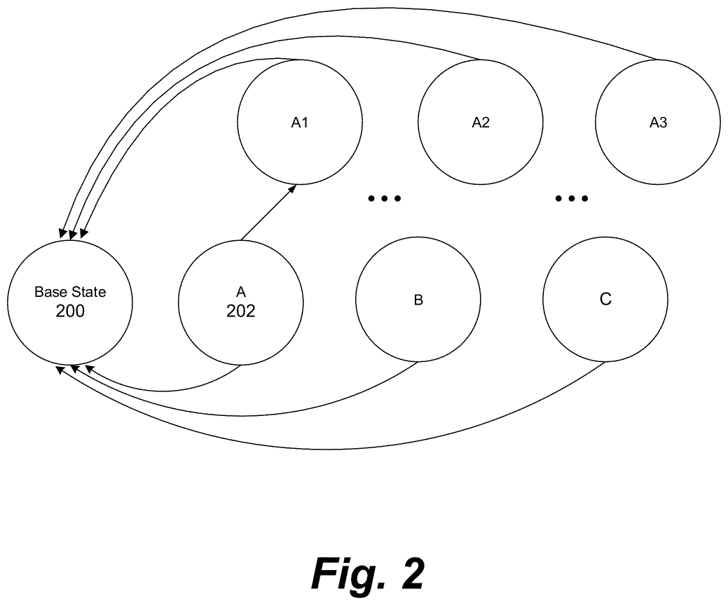

FIG. 2 illustrates example progressions through care plan states, according to one embodiment of the disclosure. When created, an individual care plan 118 assigned to a patient 102 begins at a base state 200. As the care plan management application 140 observes sensor data, the personal care plan 118 may transition into different states if the biometrics observed in the sensor data trigger thresholds associated with the different states. For example, the care plan 118 may transition from the base state 200 to state “A” 202 if thresholds associated with state “A” 202 are triggered.

The different states may branch off from a current state based on the observed biometrics. For example, depending on subsequently observed biometrics, the state “A” 202 may transition into either a state “A1” or a state “B”. Each subsequent state may branch off into multiple states. Furthermore, if subsequently observed biometrics indicate an improvement, i.e., the biometrics fall below a previously triggered threshold, then the personal care plan 118 may revert to a previous state. As the care plan 118 transitions into another state, the care plan management application 140 modifies the care plan 118, e.g., by adding, changing, or removing tasks and thresholds as specified by an event rule associated with the state.

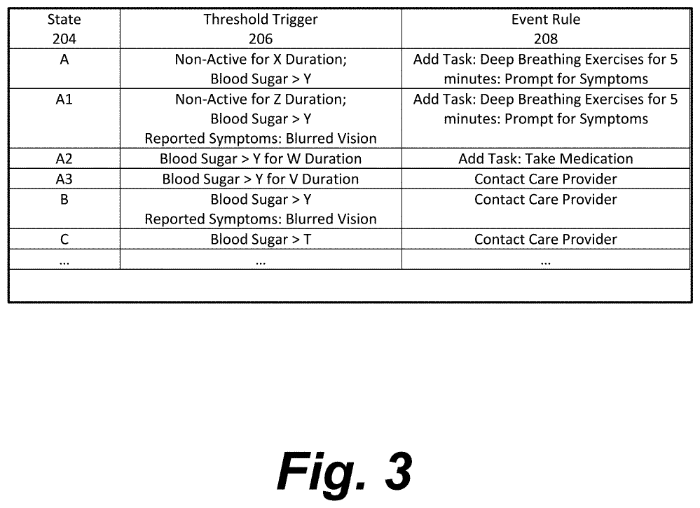

FIG. 3 illustrates a table listing states associated with each threshold and event rule, according to one embodiment. As shown, the table includes a state column 204, a threshold triggers column 206, and a event rules column 208. The state column 204 lists the states depicted in FIG. 2. The threshold triggers column 206 lists conditions that observed metrics must satisfy to trigger a threshold of a given state. For example, a threshold for state A specifies that the observed metrics should indicate that the patient 102 is currently sedentary and the patient's heart rate is greater than a value Y. If so observed, the individual care plan 118 transitions from a current state to an A state.

The event rules column 208 lists tasks for the care plan management application 118 to add, remove, or modify to the personal care plan 118 in response to observed biometrics triggering a threshold. For example, if the observed metrics triggers thresholds associated with state A, then the personal care plan 118 adds a task instructing the patient to perform deep breathing exercises for five minutes. Added tasks generally aim for reorienting observed metrics towards a desired result. The event rule may also specify that the care plan management application 140 insert new thresholds, e.g., thresholds associated with branching states, to the care plan.

In one embodiment, prior to transitioning to a new state, the care plan management application 140 may require approval from a care provider 104. For example, assume a given event rule specifies a task to be added to the personal care plan 118 that instructs the patient 102 to take a higher dosage of a given medication. Generally, instructions to alter a prescribed dosage of a given medication (or to change to an entirely different medication) require authorization from the care provider 104. Accordingly, the care plan management application 140 may first notify the care provider 104 of the state change and the proposed task prior to adding the task to the personal care plan 118. For example, the care plan management application 140 could detect that a particular prescribed medication is not successfully treating a patient's condition, e.g., due to monitored biometric data remaining outside of the target range, due to symptoms reported by the patient, and so on. In response, the care plan management application 140 could generate one or more proposed alterations to the patient's care plan, using rules corresponding to the assigned task as well as the monitored biometric data, reported symptoms and historical treatment information. The care plan management application 140 is operable to transmit these proposed alterations to the care provider 104 and to provide an interface through which the care provider 104 can select and authorize one of the proposed alterations to the patient's care plan. Additionally, the care plan management application 140 allows the care provider 104 to customize the selected care plan alteration.

Once the care plan management application 140 receives authorization for one of the proposed care plan alterations (and any customization of the selected alteration) from the care provider 104 to add the task, the care plan management application 140 adds the task to the personal care plan 118. Further, in one embodiment, prior to transitioning to a new state, the care plan management application 104 may prompt the care provider 104 to specify tasks to add or to select a task from a list of suggested tasks to add to the personal care plan 118.

Some states may correspond to end states indicating that tasks added to the personal care plan 118 are ineffective at improving the patient's biometrics. If the personal care plan 118 transitions into an end state, then an associated action for the care plan management application 140 may be to contact a care provider or emergency services to further assist the patient. Illustratively, states A3 and C represent end states.

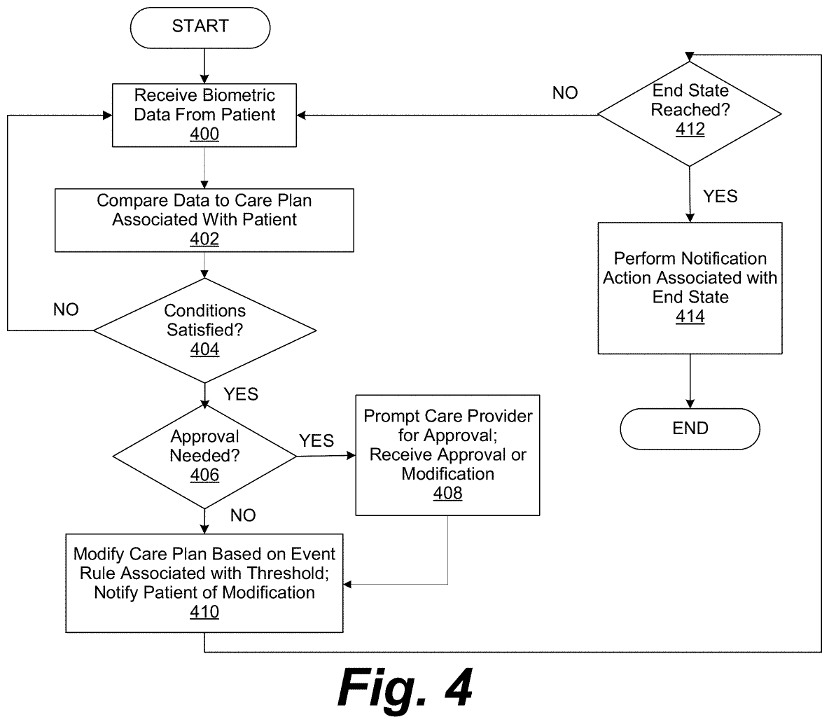

FIG. 4 illustrates a generalized method for modifying a care plan assigned to a patient, according to one embodiment. At step 400, the care plan management application 150 begins receiving data from a patient. The data provides biometrics of the patient observed through a variety of sensor devices 112a-n. The observed biometrics may include heart rate, weight, blood sugar level, whether the patient is in an active or sedentary state, and so on. In addition, the data can provide symptoms reported to the care plan management application 140 via the personal patient care application 118.

At step 402, the care management application 140 evaluates the sensor data and reported symptoms against the personal care plan 118 assigned to the patient. At step 404, the care management application 140 determines whether a condition specified in the personal care plan 118 has been satisfied. If not, the care management application 140 returns to step 402 and continues to receive sensor data from the patient. However, if a given condition is satisfied, the care plan management application identifies an event rule associated with the condition. If any actions specified in the event rule require care provider approval (at step 406), then the care plan management application 140 notifies a care provider 104 at step 408 and prompts the care provider 104 for approval for customization to be added to the tasks. At step 410, the care management application 140 performs the actions specified in the event rule (or actions specified by the care provider). At step 412, the care management application 140 determines whether an end state has been reached. If an end state has not been reached, processing returns to step 400. If, however, and end state has been reached, processing proceeds to step 414 and a notification is generated to indicate that processing has ended.

As discussed above, the invention of the present disclosure comprises a plurality of nodes for implementing the individual patient care plan. The discussion below provides a functional description of various aspects of those nodes.

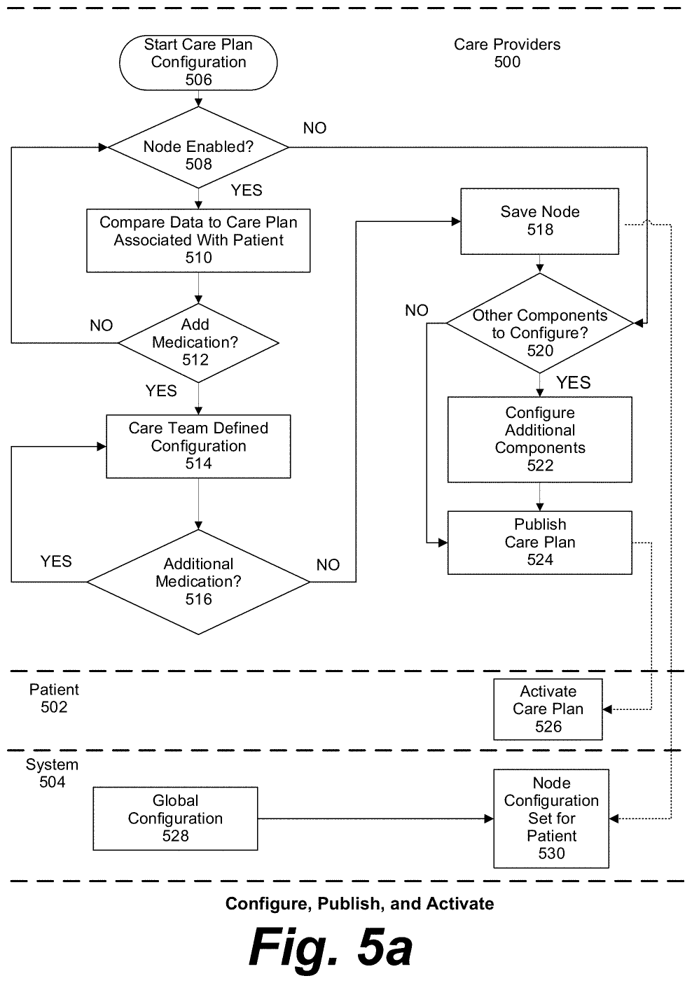

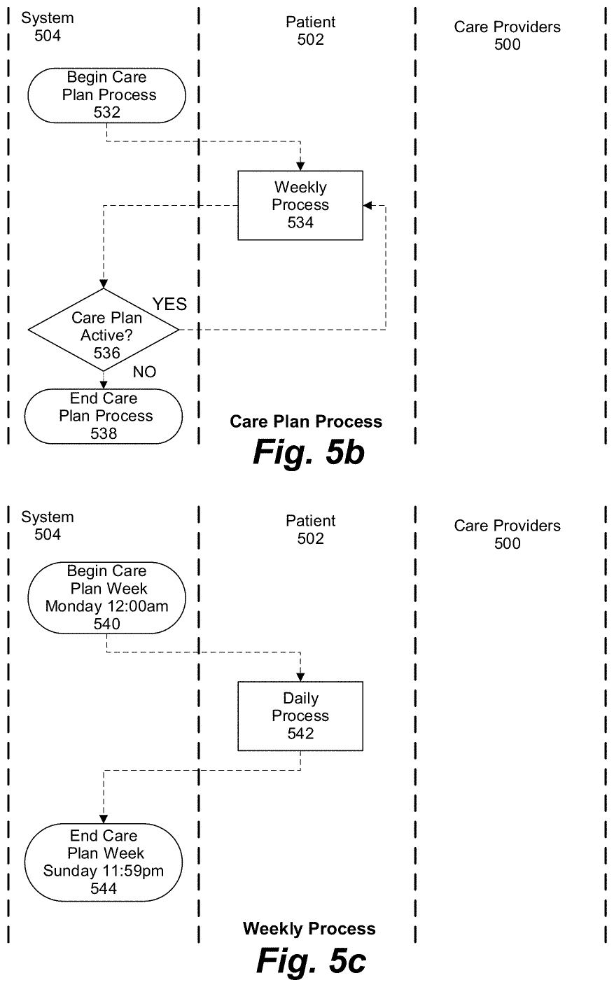

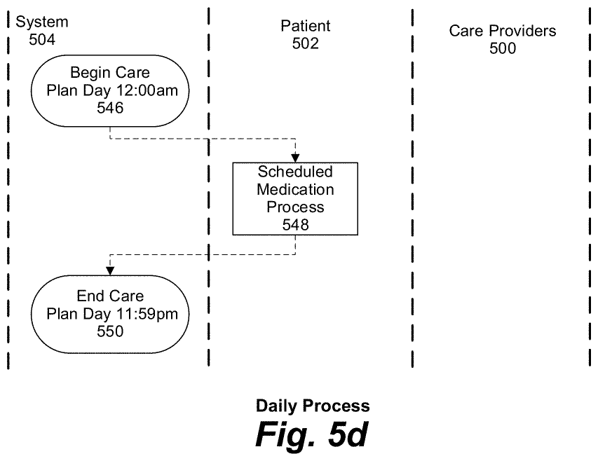

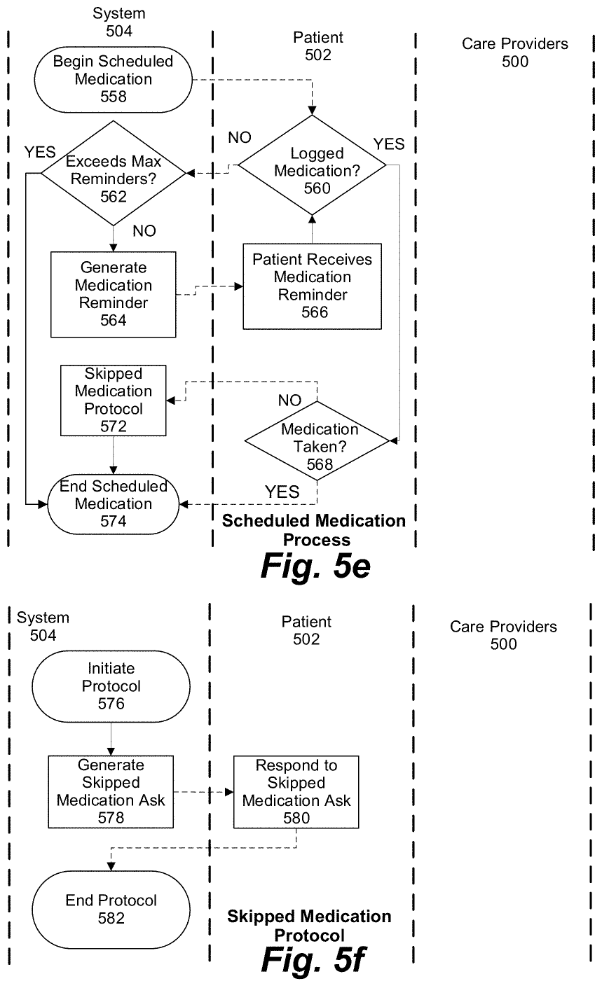

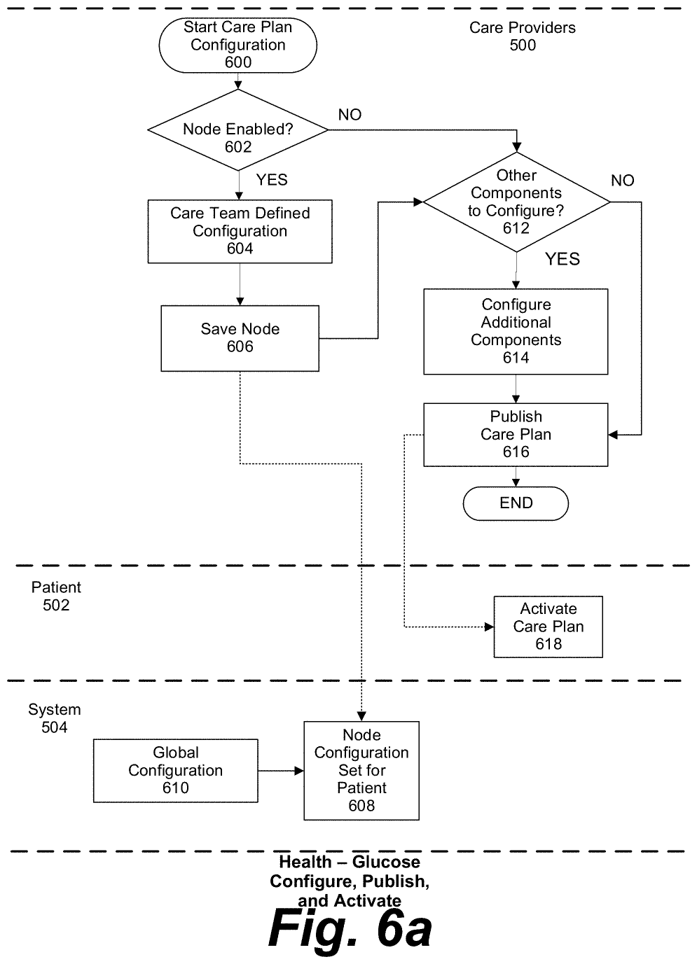

FIG. 5a is a flow chart illustration of processing steps to configure, publish, and activate a process for a care plan for the Medication—Medicine node of a Type II Diabetes care plan (hereinafter referred to as the “Medicine” node). The Medicine node is used to log and track the medication intake of a patient. During the care plan configuration, a care team member is prompted to configure several values for the Medication node. The care team use may add a predetermined number of medications per patient at any time during the care plan in the Medicine node. For each medicine, there are the following configuration fields: 1) Medication Name & Dosage; 2) Frequency; 3) Reminder, 4) Start Date; 5) End Date; 6) Take with Meal; and 7) Time Sensitive.

Medication Name & Dosage is a text field that allows the care team member to define the name, and include any relevant dosage information, for the medication added. The field is type-to-text with a set number of glucose-managing medications pre-populated in the system for convenience. Upon setting the Frequency, the care team member will be able to set the Reminder time(s). If the Frequency is set to Per Day, the Reminder values can be defined only for a given time such as “8:30 am”. The “pick list” options are in 30-minute increments spanning the 24 hours of a day. For example, the options are: 12:00 am, 12:30 am, . . . , 11:00 pm, and 11:30 pm. If the Frequency is set to Per Week, the Reminder value can be defined for both a given time and day, such as “8:30 am” on “MON”. The pick list options are “MON”, “TUE”, “WED”, “THU”, “FRI”, “SAT”, and “SUN” for each day of the week.

“Start Date” and “End Date” are date fields with calendar helpers allow the care team member to define the start and end dates for the medication added. For “Start Date,” instead of defining a date, the care team member can simply select an “Immediately” checkbox, which will default the current date. For “End Date,” instead of defining a date, the care team member can simply select a “None” checkbox, which will keep the medication active throughout the care plan.

“Take with Meal” is a variable that allows the care team member to define if the medication added is required to be taken with food. It drives labels and messaging within the app to help remind the patient to take their medication with a meal. “Time Sensitive” is a variable that allows the care team member to define if the medication added is required to be taken at specific times. It drives labels and messaging with the app to help reminder the patient to take their medication close to the scheduled time.

The Medication node has several global configuration values that are preset within the care plan template that cannot be viewed or modified by the care team. They consist of the following: 1) Max Scheduled Missed Medicine; 2) Reminder Interval Missed Medicine; 3) Percent Medication Compliance; 4) Max Scheduled Missed Medicine is the number of times that follow up reminders are sent for a missed medication. The default value is defined as “1”.

“Reminder Interval Missed Medicine” is the interval of time in minutes between the initial reminder and follow up reminders for a missed medication. “Percent Medication Compliance” is the percentage of times that a patient must take their medications per the aggregated Frequency to be considered compliant.

The specific steps of the process shown in Figure Sa will now be discussed. In step 506, configuration of the medication component of the care plan begins and processing proceeds to decision point 508, where a determination is made regarding whether the respective node is enabled. If the result decision point 508 indicates that the respective node is not enabled, processing proceeds to step 520 where a determination is made regarding whether other components of the system need to be configured. If the decision in step 520 indicates that no other components are to be configured, processing proceeds to step 524 where the care plan is published and is available for the Patient 502 to activate in step 526.

Returning to discussion of step 508, if the result of processing at that decision point indicates that the node is enabled, processing proceeds to step 510 where the proposed medication data is compared to the existing medication data of the care plan is associated with the patient. At decision point 512, the results of the processing in step 510 are analyzed and a decision is made regarding whether to add additional medication to the patient's care plan. If a determination is made that medication should be added to the care plan, then a care team defined configuration is implemented and processing proceeds to step 516 where a decision is made again regarding whether to add additional medication to the care plan. If the decision in step 516 is “YES,” processing returns to step 514. If, however, the decision in step 516 is “NO,” processing proceeds to step 518 where the configuration of the node is saved. The global configuration module 528 of System 504 also provides a global configuration update of the patient node update 530.

Once the patient has activated their personalized care plan on their mobile device, their associated care plan duration clock begins. They are then ready to begin logging their medication per the designated “Frequency” parameter of each medication. The patient is expected to continue logging a medication as long as it is “active,” with “today” being defined as a date after the Start Date and prior or equal to the End Date. FIG. 5b is a flow chart illustration of the Care Plan Process for patients to log adherence activity for the medication component of their personal care plan. The process begins in step 532 after the patient 502 activates the personal care plan as discussed above in connection with FIG. 5a. The patient 502 follows instructions on a mobile device, wherein the instructions correspond to the personalized care plan for patient 502. During periods of activity, the decision point 536 continuously monitors inputs received from the app 116 on the patient's mobile device 114 to confirm that the patient 502 is undertaking processes in the personal patient care plan 118. When the result of the processing in decision point 536 indicates that patient activity has ceased, processing ends in step 538.

FIG. 5c is a flow chart illustration of the weekly care plan process for patients to log adherence activity for the medication component of their personal care plan. The process begins in step 540 after the patient 502 activates the personal care plan as discussed above in connection with FIG. 5b. The care plan week begins each Monday at 12:00 am and ends at 11:59 pm on Sunday. The patient 502 follows instructions and executes the daily care plan processes, as discussed above in connection with FIGS. 5a-b, wherein the instructions correspond to the individual care plan 118 for patient 502. During periods of activity, the decision point 536 continuously monitors inputs received from the app on the patient's mobile device to confirm that the patient 502 is undertaking processes in the personal patient care plan.

FIG. 5d is a flow chart illustration of the daily care plan process for patients to log adherence activity for the medication component of their personal care plan. The process begins in step 540 after the patient 502 activates the personal care plan 118 as discussed above in connection with FIG. 5b. The care plan week begins each day at 12:00 am and ends at 11:59 pm on Sunday. The patient 502 follows instructions and executes the daily care plan processes, as discussed above in connection with FIGS. 5a-b, wherein the instructions correspond to the personal care plan 118 for patient 502.

FIG. 5e is a flow chart illustration of the Scheduled Medication process for monitoring patient adherence with the medication component of an personal care plan. For each scheduled medication, the patient will be able to self-report, or log, if the medication was taken. If the patient does not log the medication as taken or skipped, the patient will receive a reminder, per the scheduled Frequency and Reminder parameters asking them to confirm if the medication was taken or skipped. If the patient does not log the medication as taken or skipped either by self-reporting or via reminders, it is assumed that the medication was skipped. Up to 48 hours after the medication was scheduled, which will always include “today” and “yesterday,” the patient can update their response, or lack thereof.

There are four different reminders types that are dependent on the “Take with Meal” and “Time Sensitive” configuration settings for each medication. If the medication does not have “Take with Meal” and does not have “Time Sensitive” checked, then the patient will receive a no restrictions medication reminder. If the medication does have “Take with Meal,” but does not have “Time Sensitive” checked, then the patient will receive a “Take With Food” medication reminder. If the medication does have “Time Sensitive” checked, but does not have “Take with Meal” checked, then the patient will receive a “Time Sensitive” medication reminder. If the medication does have “Take with Meal” checked, and does have “Time Sensitive” checked, then the patient will receive a “Meal and Time Sensitive” medication reminder

As shown in FIG. 5e, the “Scheduled Medication” process begins in step 558 after the patient 502 activates the personal care plan on a mobile device and the System 504 executes the scheduled medication monitoring process. In the decision point 560, the System 504 determined whether the Patient 502 has logged data indicating compliance with the patient's personal care plan. If the processing at decision point 560 indicates that the patient has not complied with a medication requirement, processing proceeds to decision point 562 where the System 504 determines whether the patient has already received the maximum allowed number of reminders. If the processing at decision point 562 indicates that the patient has already received the maximum number of reminders, the system proceeds to step 574 and the process is ended. If, however, the processing in step 562 indicates that the patient 502 has not received the maximum number of reminders, processing continues in step 564 and a medication reminder is generated and sent to the patient. In step 566, the patient receives the reminder and processing returns to decision point 560 where the system 502 determines whether the patient 502 has provided a log entry indicating that the medication has been taken. If the result of the processing at decision point 560 indicates that the patient has not provided a log entry indicating that the medication has been taken, processing proceeds to step 572 where the System 502 initiates the ‘Skipped Medication Protocol’ and the ‘Skipped Medication Process’ is ended in step 574. If, however, the processing at decision point 560 indicates that the patient 504 has not provided a log entry indicating that the medication has been taken, processing proceeds directly to step 574.

If the patient chooses to skip a medication, either via self-reporting or responding to a reminder, they will enter the Skip Medication Protocol. The patient will be sent a follow-up message to confirm why their medication was skipped, as this is critical information for the care team to store for future reporting. FIG. 5f is a flow chart illustration of the care plan process for skipped medication protocol. In step 576, the System 502 initiates skipped medication protocol and processing proceeds to step 578 where a skipped medication “ask” is generated and sent to the Patient 502. In step 580, the Patient 502 responds to the skipped medication “ask” and processing proceeds to step 582, where the skipped medication protocol is ended.

The flowcharts and related discussion below will summarize the processing steps for other nodes. The various processes for the respective nodes include standard flowchart symbols, similar to those shown in FIGS. 5a-f; however, the processes will be discussed in a narrative form without reference to each specific flowchart process block.

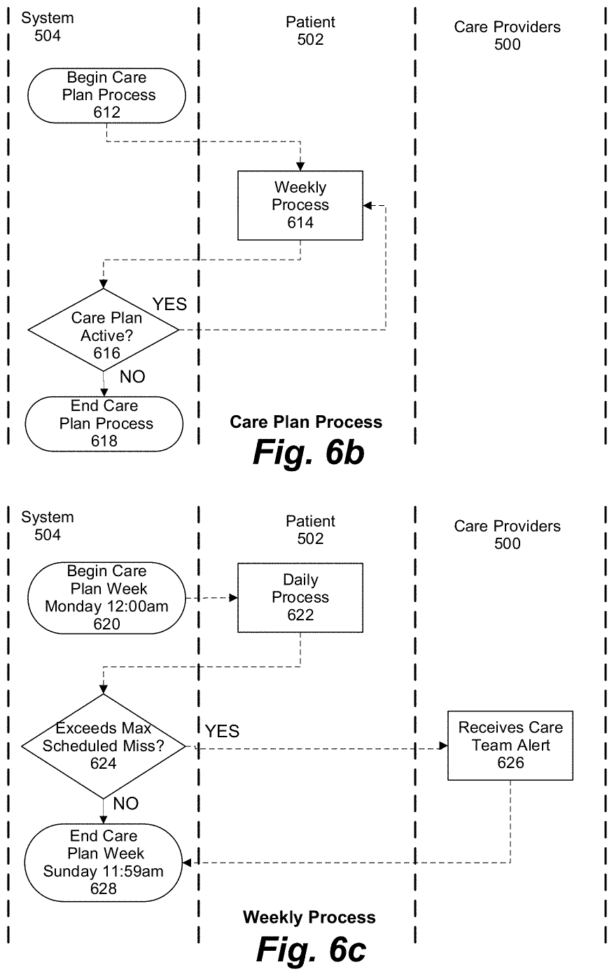

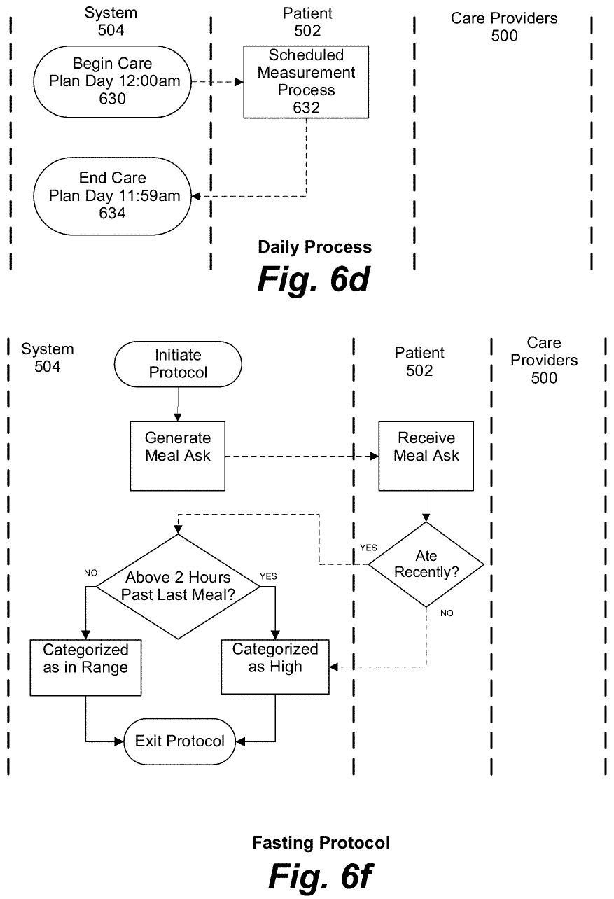

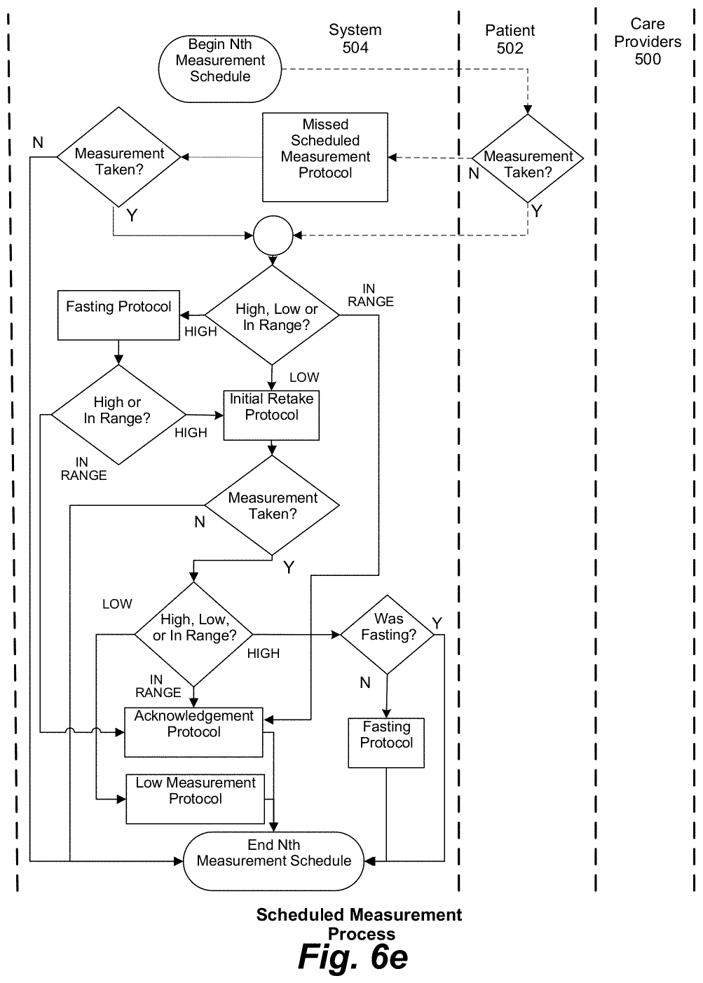

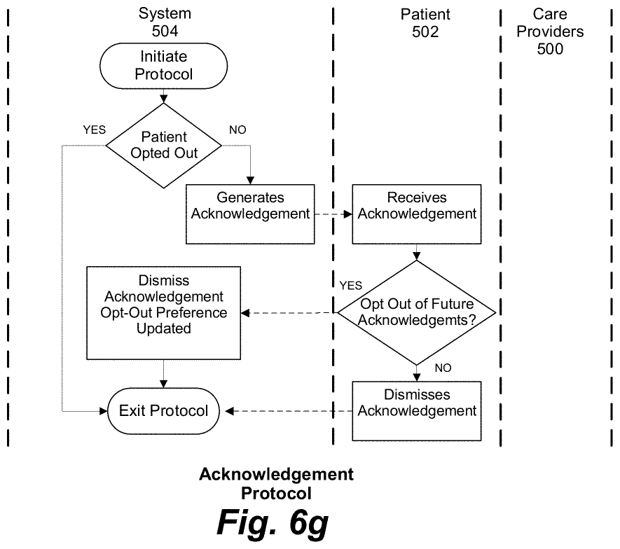

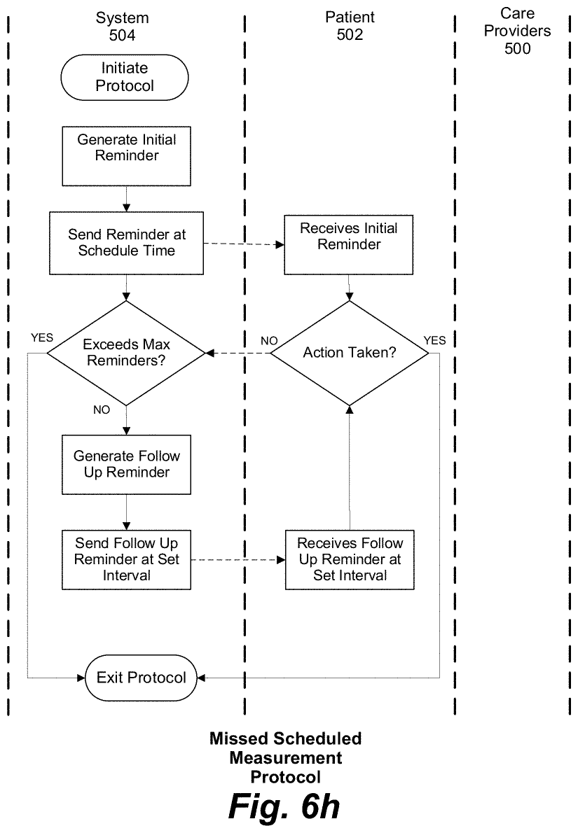

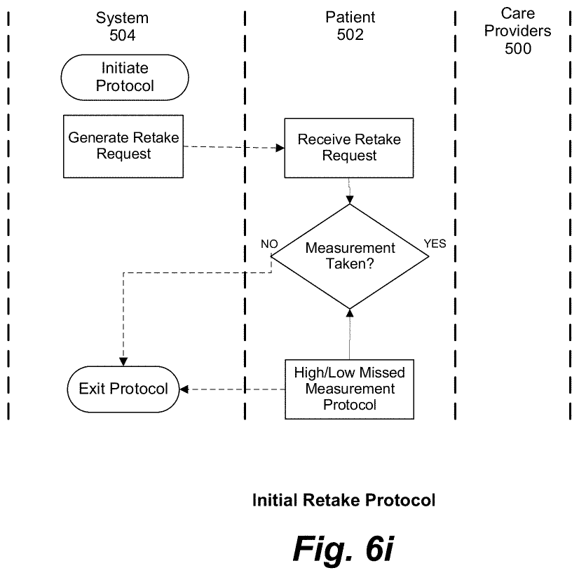

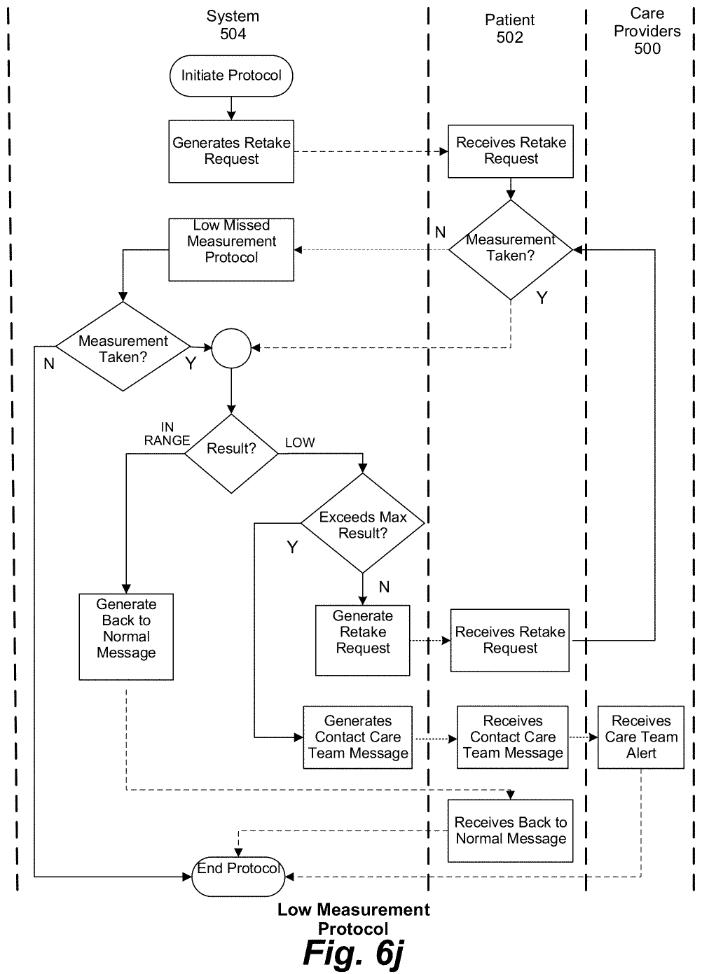

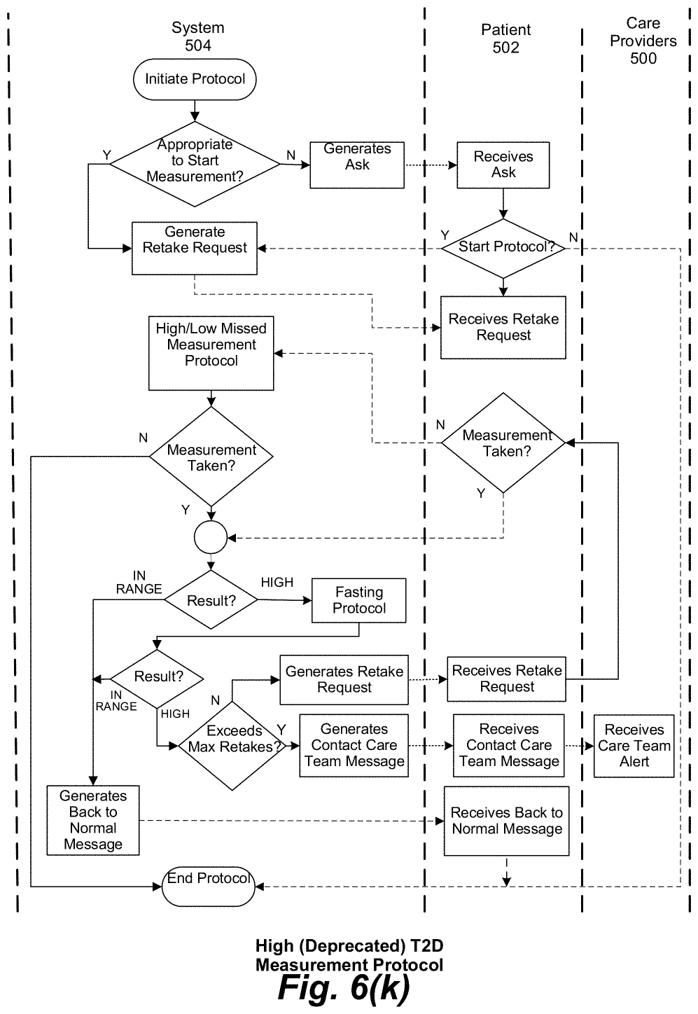

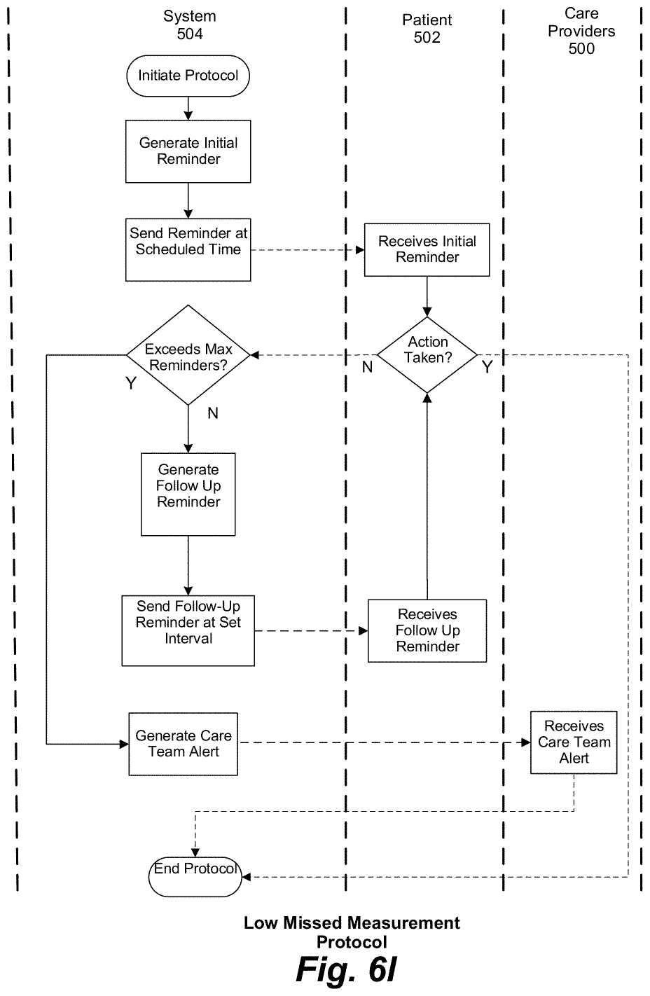

FIG. 6a is a flow chart illustration of processing steps to configure, publish, and activate a process for a care plan for the Health-Glucose node of a Type II Diabetes care plan. Before discussing the specific processing steps, a brief summary is provided for each of respective care plan processes in the flow charts.

During the care plan configuration, a care team member is prompted to configure several values for the Health-Glucose node. There are the following configuration fields: 1) Current Hemoglobin A1C Level (%): 2) Frequency; 3) Reminder; 4) Glucose Thresholds (mg/dL), including a) Low Glucose Thresholds (mg/dL) and b) High Glucose Thresholds (mg/dL)—2 Hours Post Meal.

The Current A1C Level (%) is a required numeric text field that allows the care team to define the starting A1C value for a care plan. It is the latest official value that must be retrieved from the patient's electronic medical record. Frequency is selected from a list that allows the care team to define the minimum number of times a day that the patient must take blood glucose measurements. The system automatically detects when new measurements are taken. However, if those measurements are not taken and, subsequently, detected by the system, the Frequency value is the initial driver to scheduling a Reminder. The Frequency list includes the values of “Three Times A Day”, “Twice A Day”, and “Once A Day” with “Once A Day” as the default value.

Upon setting the Frequency, the care team member will be able to set the Reminder time(s) which are defaulted to preset values, but can be adjusted by the care team member for each patient. The Reminder values are required and default based upon the Frequency selected by the care team member. If they choose “Three Times A Day”, the three Reminder values will default to “10:00 am”, “2:00 pm”, and “8:00 pm”. If they chose “Twice A Day”, the two Reminder values will default to “2:00 pm” and “8:00 pm”. If they chose “Once A Day”, the one Reminder value will default to “8:00 pm”. The pick list options for Reminder are in 30-minute increments spanning the 24 hours of a day. For example, the options are: 12:00 am, 12:30 am, . . . , 11:00 pm, and 11:30 pm.

Do Not Disturb is a set of lists that allows the care team to define the range of time for which non-life-threatening protocols may be suspended. The default values for the range are “11:00 pm” and “7:00 am” respectively. The pick list options for the Do Not Disturb fields are in 30-minute increments spanning the 24 hours of a day. For example, the options are: 12:00 am, 12:30 am, . . . , 11:00 pm, and 11:30 pm.

The Glucose Thresholds (mg/dL) fields are the three glucose thresholds that are defaulted, but the care team has the ability to edit the values. There is one low threshold, and then two high thresholds. The acceptable values for each threshold range from “50” to “200”. The Glucose Thresholds (mg/dL)—Low measurement indicates the low boundary of an “In Range” glucose measurement. The default value for this parameter is “70”. Glucose Thresholds (mg/dL)—High: This measurement indicates the high boundary of an “In Range” glucose measurement when fasting (have not eaten within the past 2 hours). It is defaulted to “130”. “Glucose Thresholds (mg/dL)—2 Hours Post Meal” is a measurement that indicates the high boundary of an “In Range” glucose measurement when not fasting (have eaten within the past 2 hours). Since blood glucose levels rise immediately after a meal, the two hours post meal glucose measurement indicates how well the patient is metabolizing sugar. The default value for this parameter is “180”.

The Glucose node has several global level configuration values that are preset within the care plan template that cannot be viewed or modified by the care team. They consist of the following: Max Retries Per Scheduled Glucose; Reminder Interval Missed Glucose; Max Reminder Missed Glucose; Max Percentage Missed Glucose; Low Glucose Frequency; High Glucose Frequency; and Glucose Percent Target Met.

Max Retries Per Scheduled Glucose is the number of times a “Low” or “High” measurement can be retested before an alert to the care team is sent. The default value for this parameter is “2”. Reminder Interval Missed Glucose is the interval of time in minutes between the initial reminder and follow up reminders for a missed measurement. The default value for this parameter is “15”. Max Reminder Missed Glucose is the number of times that follow up reminders are sent for a missed measurement. The default value for this parameter is “I”. Max Percentage Missed Glucose is the percent of regularly scheduled measurements that must be missed during a weekly period to send an alert to the care team. The default value for this parameter is “60”. Low Glucose Frequency is the number of minutes out to schedule a new one-time measurement for the Low Measurement Protocol. The default value for this parameter is “15”. High Glucose Frequency is the number of minutes out to schedule a new one-time measurement for the High Measurement Protocol. The default value for this parameter is “240”. Glucose Percent Target Met is the percent of times that glucose measurements taken by the patient must be “In Range” during a week, per their Frequency, to be considered compliant. The default value for this parameter is “80”.

The processing steps to set up the Health-Glucose node are shown in FIG. 6A. These processing steps are essentially identical to those discussed above in connection with FIG. 5A.

FIG. 6b is a flow chart illustration of the Care Plan Process for patients to log adherence activity for the Health-Glucose component of their personal care plan. Once a patient has activated their personalized care plan on their mobile device in step 618, their care plan duration clock starts. To begin tracking their blood glucose, they simply have to use their connected glucometer device the week, in accordance with their personal weekly process and take regular measurements each day to have their measurements captured

FIG. 6c is a flow chart illustration of the weekly care plan process for patients to log adherence activity for the Health-Glucose component of their personal care plan. The weekly timeframe for the Glucose node is set to start on Monday morning at 12:00 am and end on Sunday night at 11:59 pm for all patients. At the end of each week, a calculation is conducted to determine if the patient has missed more than the maximum percentage of allowed regularly scheduled glucose measurements per the Max Percentage Missed Glucose global configuration value. If they have, an alert will be sent to the care team.

At weekly intervals, a calculation will be done to determine the adherence percentage based on number of patients provided glucose measurements that were in range out of the weekly sum of regularly scheduled measurements. If they have achieved the Glucose Percent Target Met, they will be considered in adherence.

FIG. 6d is a flow chart illustration of the daily care plan process for patients to log adherence activity for the Health-glucose component of their personal care plan. The daily timeframe for the Glucose node will be set to start early in the morning at 12:00 am and end late at night at 11:59 pm for all patients. Throughout the day, the patient is expected to take blood glucose measurements per the Frequency configured in their care plan as documented in the Scheduled Measurement Process. For a Frequency of “Three Times A Day”, the first measurement should be taken prior to the time defined for their first Reminder, the second measurement should be taken after their first Reminder prior to their second Reminder, and the third measurement after their second Reminder and prior to their third Reminder. For a Frequency of “Twice A Day”, the first measurement should be taken prior to the time defined for their first Reminder, and the second measurement should be taken after their first Reminder prior to their second Reminder. For a Frequency of “Once A Day”—the one measurement should be taken prior to the time defined for the Reminder.

FIG. 6e is a flow chart illustration of the Scheduled Measurement component of the Health-Glucose component of a the patient's personal care plan. For each scheduled measurement, the patient is required to proactively take the glucose measurement. If the patient does not, they will receive a reminder per the Frequency and Reminder schedule prompting them to take a glucose measurement. If the patient still does not, it is categorized as missed. If the patient provides a glucose measurement, they will be directed into the Acknowledgement Protocol or the Initial Retake Protocol based on their response

FIG. 6f is a flow chart illustration of the care plan process for the Fasting Protocol for the Health-Glucose Glucose component of their personal care plan. If a glucose measurement exceeds the High threshold, the patient will be prompted with an urgent ask to confirm if they were fasting prior to taking that measurement. Fasting for this scenario means that, prior to the measurement, the patient had not eaten within the past two hours. After the patient answers this question, the calculations will be rerun per the Fasting Protocol. If the patient confirmed that they were fasting, they will have exceeded the normal High threshold. If the patient confirmed that they were not fasting, then an additional calculation will be run to see if they have exceeded the Two Hours Post Meal threshold. If the not fasting scenario ends up resulting in a value that falls below the Two Hours Post Meal threshold, then it is categorized as “In Range” and no further action is taken.

FIG. 6g is a flow chart illustration of the Acknowledgement protocol component of the Health-Glucose component of a the patient's personal care plan. If a glucose measurement is “In Range”, above the Low threshold and below the High or Two Hours Post Meal thresholds depending on fasting status, the patient will be sent an acknowledgement confirming that the measurement was received by the system. If the patient does not want to receive future acknowledgements, they can opt-out.

FIG. 6h is a flow chart illustration of the Missed Scheduled Measurement component of the Health-Glucose component of a the patient's personal care plan. If the patient misses a regularly scheduled glucose measurement, they will receive a series of reminders per the global configuration values to take a blood glucose measurement. If the patient does not reply, or chooses to skip the scheduled measurement, the care team will not be sent an alert at that time.

FIG. 6i is a flow chart illustration of the Initial Retake Protocol of the Health-Glucose component of a the patient's personal care plan. Upon providing a High or Low measurement, the patient will be prompted to take another glucose measurement for confirmation. Depending on if the patient takes another measurement and the result of it, the patient will enter the High/Low Missed Measurement, High, Low, or Acknowledgement protocols

FIG. 6j is a flow chart illustration of the Low Measurement Protocol of the Health-Glucose component of a the patient's personal care plan. Once the patient has confirmed a “Low” glucose measurement with another “Low” glucose measurement via the Initial Retake Protocol, the patient enters the Low Measurement Protocol. After taking action to increase their blood glucose levels, if the patient provides an “In Range” measurement, then the patient will exit the Low Measurement Protocol while receiving a message on how to avoid “Low” glucose level situations in the future. If the patient's follow up blood glucose measurement is still “Low”, the patient will receive a message, and another test will be scheduled if they have not exceeded the Max Retries Per Scheduled Glucose global configuration value. This process will repeat until the patient provides an “In Range” measurement, or they have exceeded the Max Retries Per Scheduled Glucose global configuration value. If the subsequent measurement is “In Range”, no further action is needed, and the patient will receive a message confirming it. If the glucose measurement is still “Low” on the subsequent test, and they have exceeded the Max Retries Per Scheduled Glucose global configuration value, the patient will be prompted with a message instructing them to contact their care team, and an alert will be sent to the care team.

FIG. 6k is a flow chart illustration of the High(/Low Deprecated) Missed Measurement component of the Health-Glucose component of a the patient's personal care plan. Once the patient has confirmed a “High” glucose measurement with another “High” glucose measurement via the Initial Retake Protocol, if appropriate, the patient may enter the High Measurement Protocol. After taking action to lower their blood glucose levels, if the patient provides an “In Range” measurement, then the patient will exit the High Measurement Protocol while receiving a message confirming that they are back “In Range”. If the patient's follow up blood glucose measurement is still “High”, the patient will be prompted with a message to confirm if they were fasting during that latest measurement as all of the Fasting Protocol rules still apply for calculations and thresholds. If the latest blood glucose measurement is categorized as “High”, the patient will receive a message, and another test will be scheduled if they have not exceeded the Max Retries Per Scheduled Glucose global configuration value. If the subsequent measurement is “In Range”, no further action is needed, and the patient will receive a message confirming it. If the glucose measurement is still “High” on the subsequent test, and they have exceeded the Max Retries Per Scheduled Glucose global configuration value, the patient will be prompted with a message instructing them to contact their care team, and an alert will be sent to the care team.

FIG. 6l is a flow chart illustration of the Low Missed Measurement Protocol component of the Health-Glucose component of a the patient's personal care plan. If the patient misses a glucose measurement during the Initial Retake Protocol, the High Measurement Protocol, or the Low Measurement Protocol, they will initially receive a reminder that action is needed to provide a blood glucose measurement as soon as possible. If the patient does not reply, or chooses to skip the scheduled measurement, a critical alert will be sent to the care team.

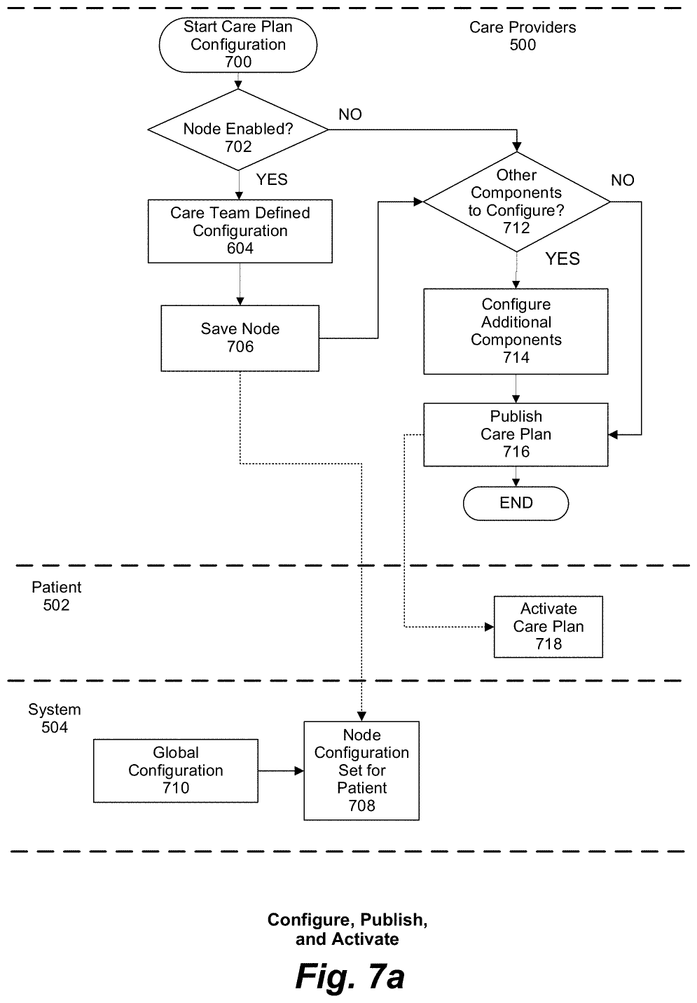

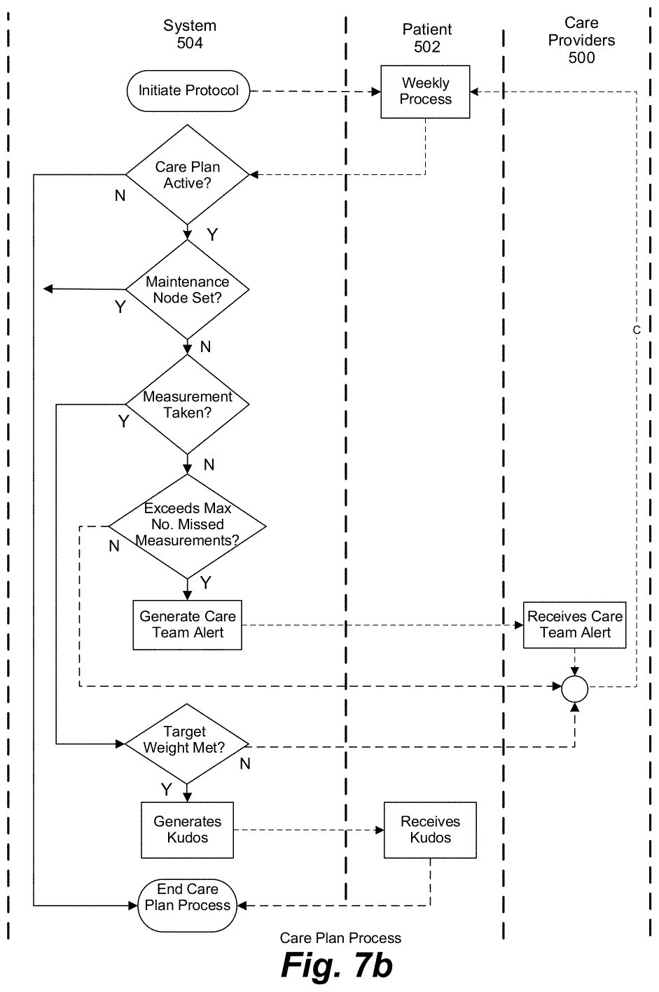

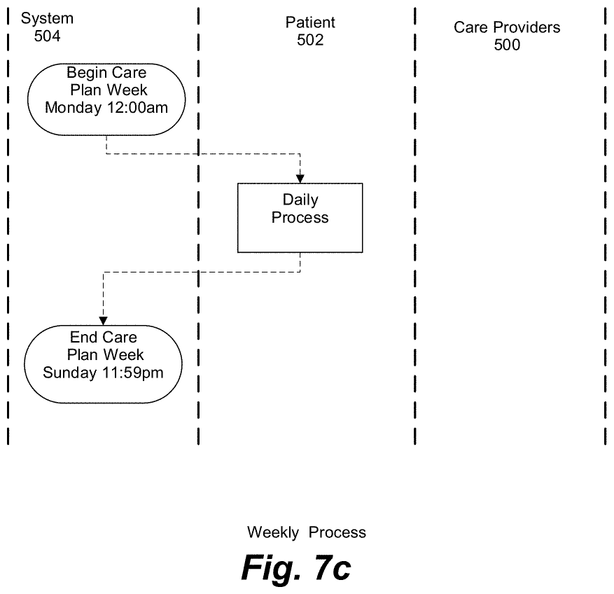

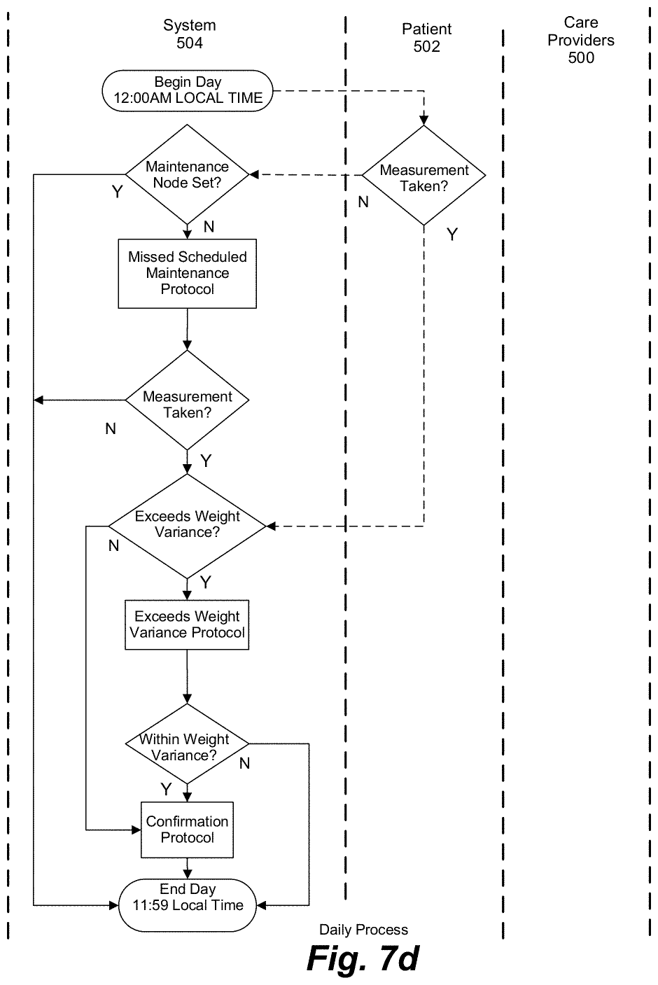

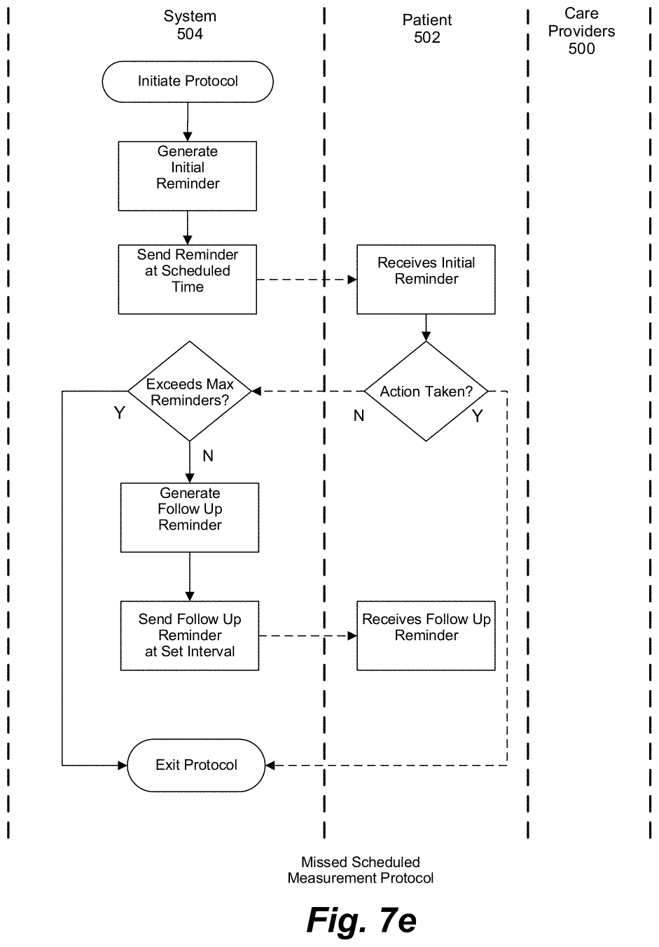

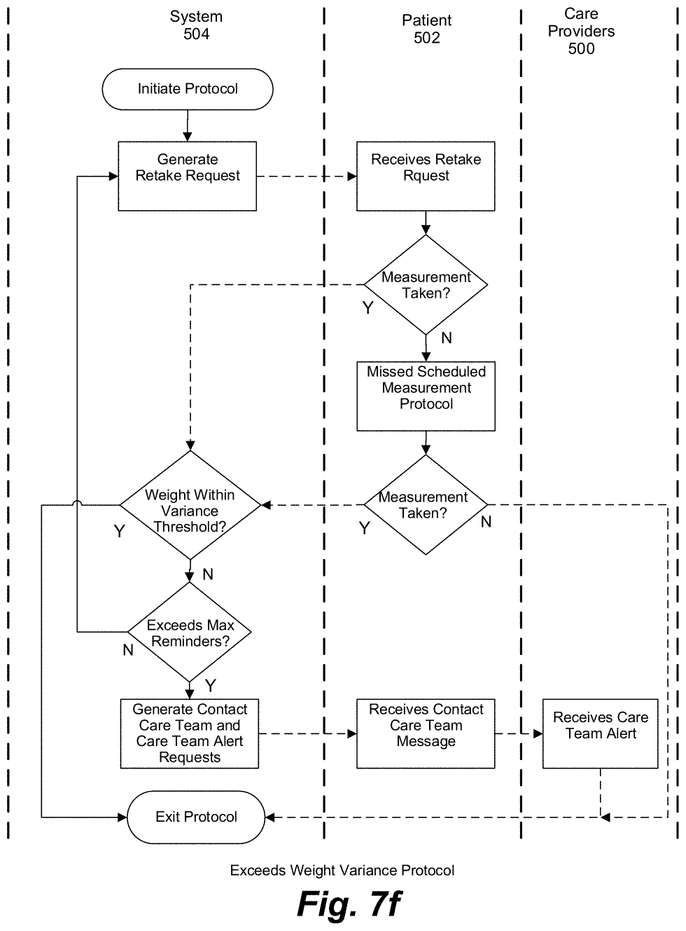

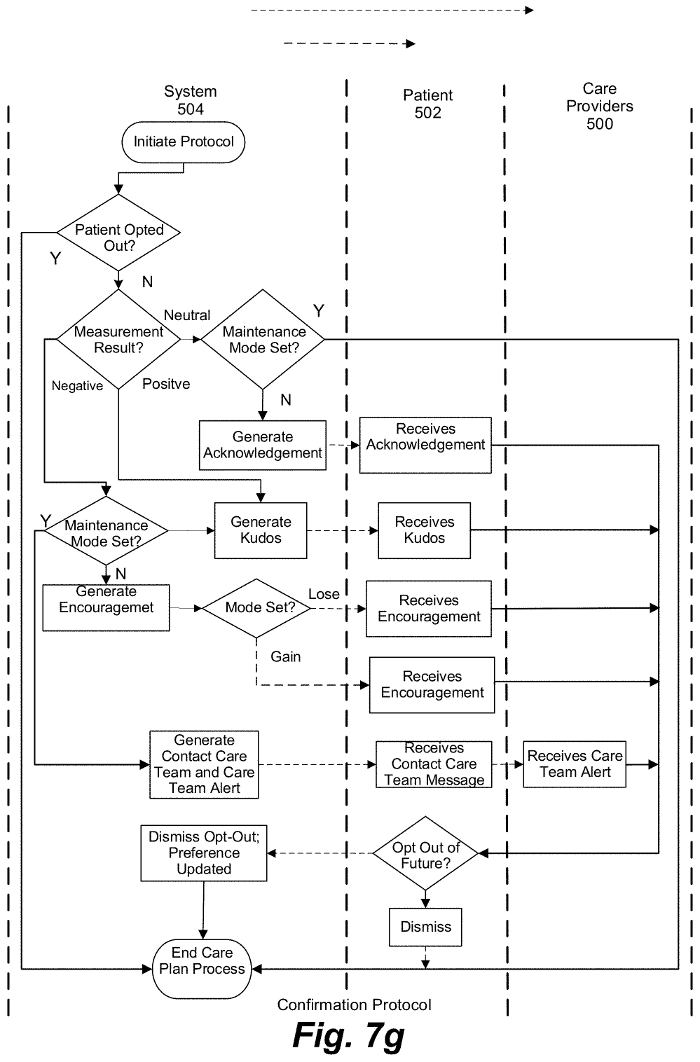

FIG. 7a is a flow chart illustration of the Configure, Publish, and Activate protocol of the Health-Weight component of a the patient's personal care plan. As part of the Health component within the Type II Diabetes Care Plan, there is a Health-Weight node that is configured and implemented for each patient. The Weight node is used to monitor the weight measurements of a patient.

During the care plan configuration, a care team member is prompted to configure several values for the Weight node. There are the following configuration fields: 1) Height; 2) Starting Weight; 3) Official Weight; 4) Weight Target Type; 5) Weekly Weight Goal; 6) Frequency; and 7) Reminder. Height is a numeric text field that allows the care team to define the patient's height in inches. It should be retrieved from the patient's electronic medical record, but is not required. If it is not provided, the Body Mass Index (BMI) for the patient cannot be calculated. This setting is on the Basic Information step. Official Weight is a numeric text field that allows the care team to define the patient's starting weight in pounds. It should be retrieved from the patient's electronic medical record, but is not required. If it is not provided during the configuration of the patient's care plan, it will automatically be determined from the first scheduled weight measurement. This setting is on the Basic Information step. Target Weight is a numeric text field that allows the care team to define the patient's target weight in pounds. For example, if a patient weights 136 lbs according to the first measurement provided, but should weigh 150 lbs, their Target Weight would be set to “150”. WeightTargetType is a dropdown pick list that allows the care team to define the direction to Target Weight. The pick list includes the values of: “Gain”, “Lose”, “Maintenance”. The default value for T2D is “Lose.” Weekly Weight Goal is a dropdown pick list that allows the care team to define the weight gain target in pounds each week, regardless of how frequently they take their weight. The overall total target weight for the patient will be a calculated value that multiplies the Weekly Weight Goal by the number of weeks in the care plan duration. The pick list includes the values of: “0.5 lbs per week”, “1 lbs per week” and “2 lbs per week”. The default value is “1 lb per week”.

Frequency is a combination of two required fields that allow the care team to define the minimum frequency for which the patient is expected to provide a weight measurement. The first field is a dropdown pick list that allows the care team to define if the frequency is “Weekly”. The second field is a dropdown pick list that allows the care team to define the day of the week for which the patient is expected to provide a weight measurement. The default value is “SUN” for Sunday.

Reminder is the time of day, per the Frequency, that the patient will be reminded to provide a weight measurement, if they have not earlier in the day. The default value is “7:00 pm”. The pick list options for Reminder are in 30-minute increments spanning the 24 hours of a day. For example, the options are: 12:00 am, 12:30 am, . . . , 11:00 pm, and 11:30 pm.

The Weight node has several global configuration values that are preset within the care plan template that cannot be viewed or modified by the care team. They consist of the following: 1) Weight Variance Threshold; 2) Max Number Exceeds Weight Variance; 3) Reminder Interval; 4) Missed Weight; 5) Max Reminder Missed Weight; 6) Max Number Missed Weight; 7) Percent Weight Compliance; and 8) Weight Maintenance Threshold.

Weight Variance Threshold is the percentage of variance from the previous measurement that determines if the patient should enter the Exceed Weight Variance Protocol. The default value for this parameter is “25”. Max Number Exceeds Weight Variance is the number of times that a weight measurement can be repeated for the Exceeds Weight Variance Protocol before the rules will trigger an urgent care team alert. The default value for this parameter is “2”. Reminder Interval Missed Weight is the interval of time in minutes between the initial reminder and follow up reminders for a missed measurement. The default value for this parameter is “15”. Max Reminder Missed Weight is the number of times that follow up reminders are sent for a missed measurement. The default value for this parameter is “1”. Max Number Missed Weight is the number of consecutive scheduled weight measurements that must be missed to send an alert to the care team. The default value for this parameter is “3”. Percent Weight Compliance is the percentage of the patient's targeted weight goal they must have achieved at any point in the care plan to be considered compliant at that time. The default value for this parameter is “60”. Weight Maintenance Threshold is the percentage of variance from the Target Weight that determines if the patient should enter the Confirmation Protocol. The default value for this parameter is “5”.

FIG. 7b is a flow chart illustration of the Care Plan Process protocol of the Health-Weight component of a the patient's personal care plan. Once a patient has activated their personalized care plan on their mobile device, their care plan duration clock starts. To begin tracking their weight, they simply have to step on their connected scale device per their Frequency to have their measurements captured. Once the patient achieves their Target Weight, the patient will receive a “kudos.” The patient will then enter a maintenance period. The patient can continue to track and monitor their weight with the mobile app, but they will no longer receive reminders until the Weight Target Type is set to Gain or Lose mode manually by the user If the patient misses a number of consecutive weight measurements on a weekly schedule, as defined per the Max Number Missed Weight global configuration variable, an alert will be sent to the care team.

FIG. 7c is a flow chart illustration of the Weekly Process protocol of the Health-Weight component of a the patient's personal care plan. The weekly timeframe for the Weight node will be set to start on Monday morning at 12:00 am and end on Sunday night at 11:59 pm for all patients.

FIG. 7d is a flow chart illustration of the Daily Process protocol of the Health-Weight component of a the patient's personal care plan. The daily timeframe for the Weight Loss node will be set to start early in the morning at 12:00 am and end late at night at 11:59 pm for all patients. At least once on the scheduled day for a Frequency of “Weekly”, the patient should take a weight measurement. If the patient does not provide a measurement prior to the configured Reminder, they will enter the Missed Scheduled Measurement Protocol. If the patient provides a weight measurement that does not exceed the Weight Variance Threshold, they will enter the Confirmation Protocol. If the patient provides a weight measurement that does exceed the Weight Variance Threshold, they will enter the Exceeds Weight Variance Protocol.

FIG. 7e is a flow chart illustration of the “Missed Scheduled Measurement” protocol of the Health-Weight component of a the patient's personal care plan. If the patient misses a regularly scheduled weight measurement, they will receive a series of reminders per the global configuration values to take a weight measurement. If the patient does not reply, or chooses to skip the scheduled measurement, the care team will not be sent an alert.

FIG. 7f is a flow chart illustration of the “Exceeds Weight Variance” protocol of the Health-Weight component of a the patient's personal care plan. If the patient's weight exceeds the overall Weight Variance Threshold in either direction, they will be asked to repeat their weight measurement, since it is possible that a person other than the patient may have stepped on the scale. The request to repeat their weight measurement will continue until the measurement is within the overall Weight Variance Threshold, or until the number of attempts exceeds the Max Number Exceeds Weight Variance global configuration value. If the patient exceeds the Max Number Exceeds Weight Variance global configuration value, they will receive a notification to troubleshoot their scale device, and an alert will be sent to the technical support care team member.

FIG. 7g is a table showing parameters for Weight Mode vs. Measurement Result for the Confirmation protocol of the Health-Weight component of a the patient's personal care plan. For each weight measurement, the patient will receive a follow-up message confirming the measurement with a specific mood and intent. If the patient achieves their Weekly Weight Goal for the week based on the measurement provided, the patient will receive a kudos. If the patient does not achieve their Weekly Weight Goal for the week but does not regress from the measurement taken the previous week, based on the measurement provided, the patient will receive an acknowledgment. If the patient does not achieve their Weekly Weight Goal for the week and regresses from the measurement taken the previous week, based on the measurement provided, the patient will receive an encouragement.

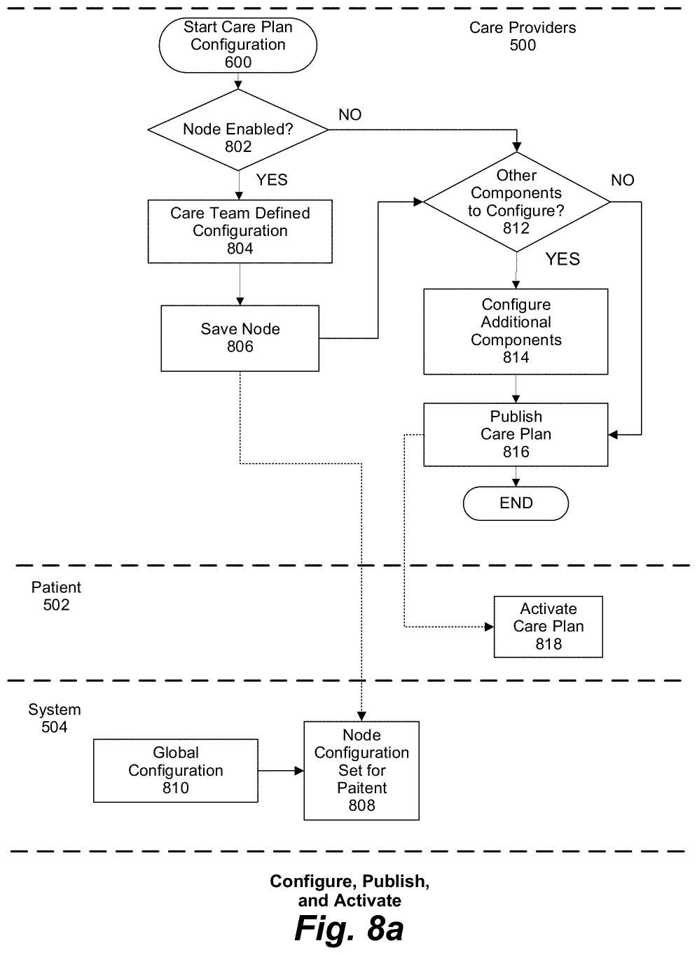

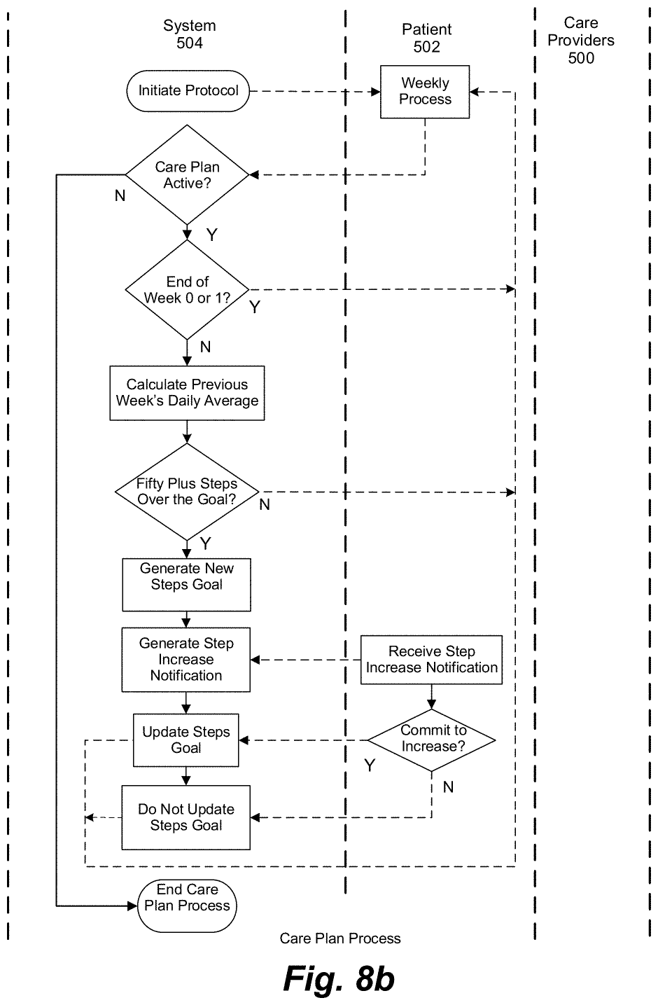

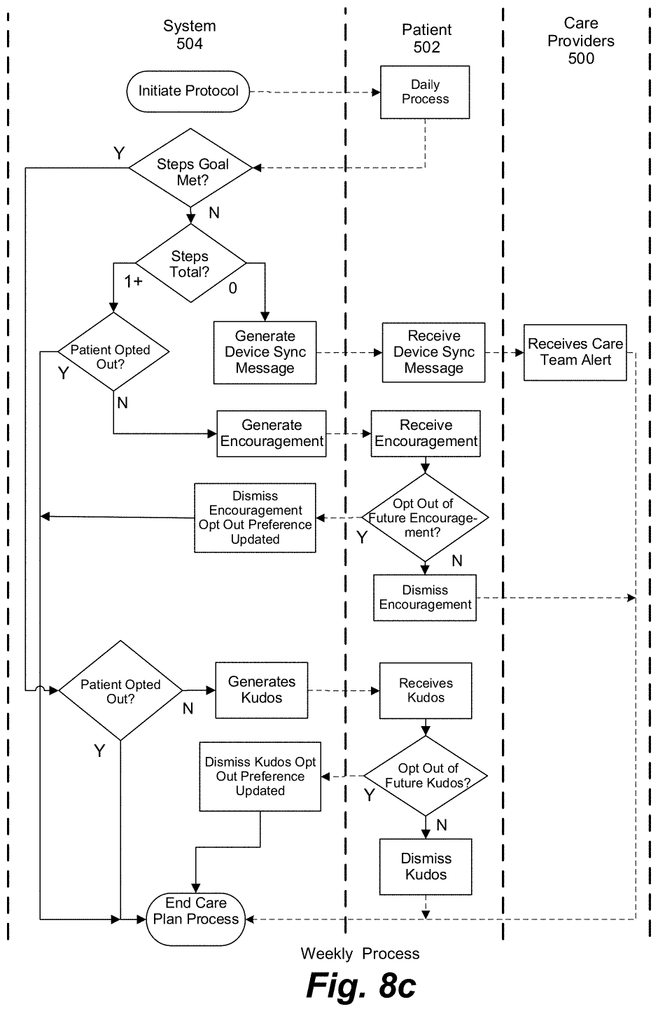

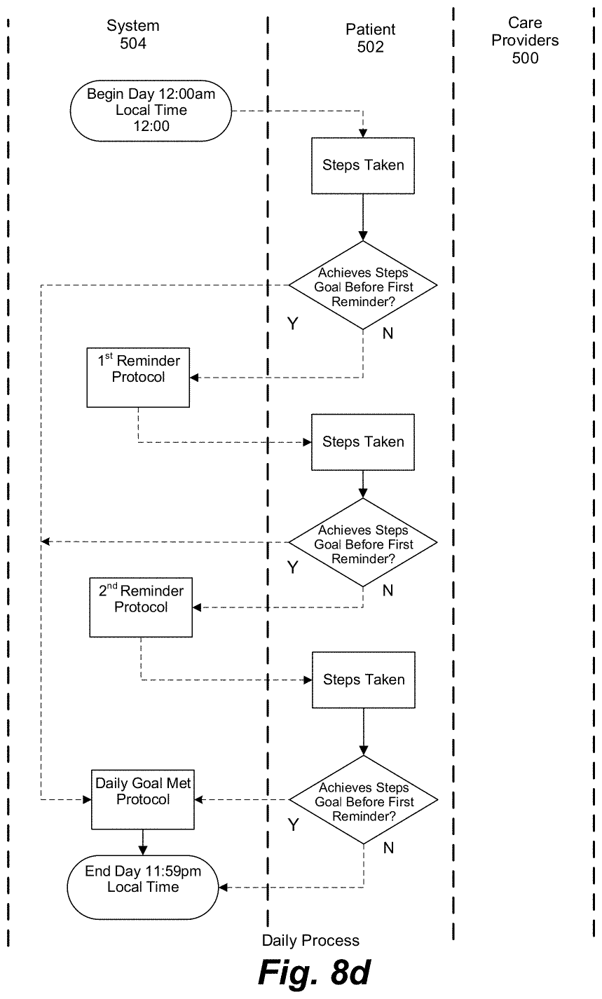

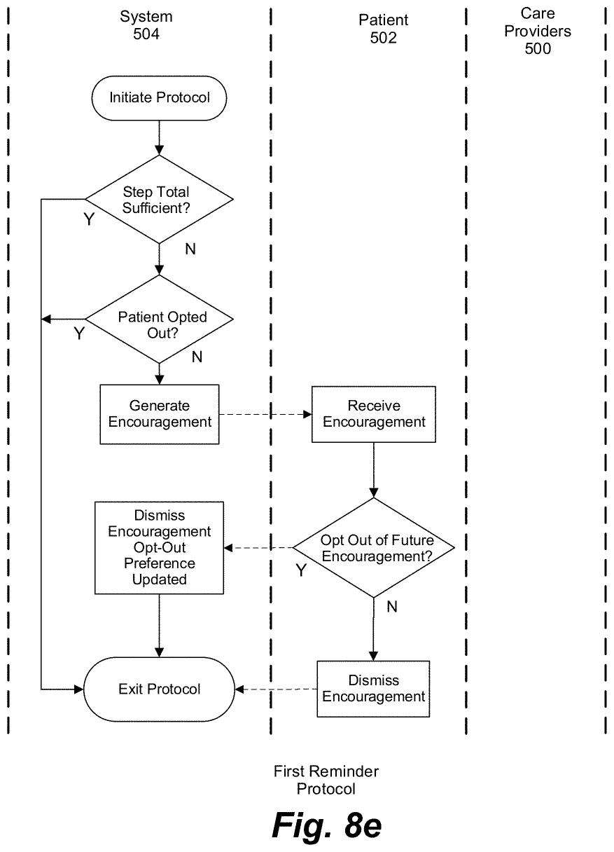

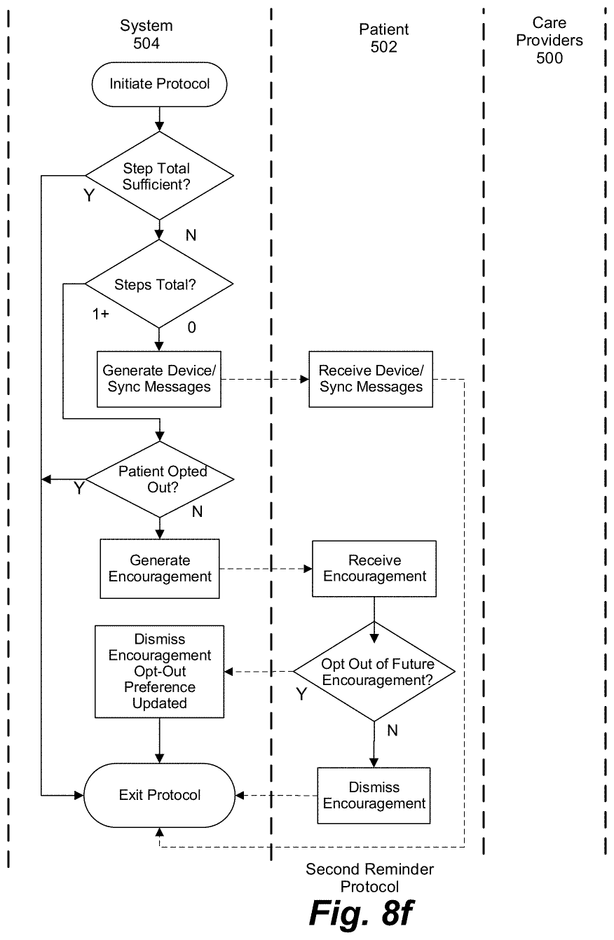

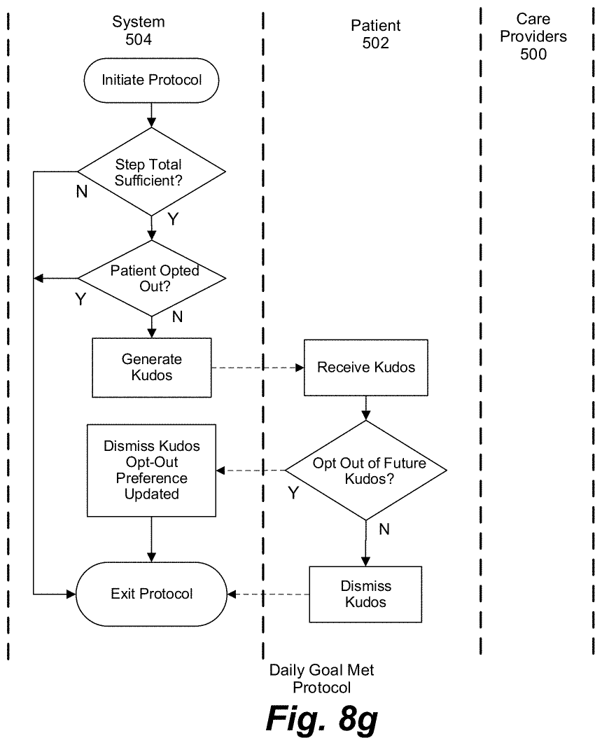

FIG. 8a is a flow chart illustration of the Configure, Publish, and Activate protocol of the Activity-Steps component of a the patient's personal care plan. During the care plan configuration, a care team member is prompted to configure the Steps node, when it is left enabled. There are the following configuration fields: 1) Activity Level; 2) Starting Daily Step Goal; 3) Current Daily Step Goal; 4) Frequency; and 5) Reminder.

Activity Level is a list with three options for the care team member to select the patient's appropriate activity level. The pick list options are: “Low Activity”, “Moderate Activity”, or “High Activity”. It will have “Moderate Activity” selected by default.

Starting Daily Step Goal is a read-only text field that displays the baseline number of daily steps that the patient will begin their care plan with for the selected Activity Level. The values for each Activity Level are: “2000” for “Low Activity”, “4000” for “Moderate Activity”, and “6000” for “High Activity”.

Current Daily Step Goal is a read-only text field that displays the daily steps goal for the patient as of the current date and time. If a patient is not on track to meet their daily steps goal, the Frequency dropdown pick list allows the care team member to define the number of times per day that the patient is encouraged to get up and become more active. The options available are “Once A Day” and “Twice A Day”. The Reminder fields are dropdown pick lists allowing the care team member to set the time of day for which the patient should be encouraged per the configured Frequency. The list options are in 30 minute increments spanning the 24 hours of a day. For example, the options are: 12:00 am, 12:30 am, . . . , 11:00 pm, and 11:30 pm. The Reminder values are required and default based upon the Frequency selected by the care team member. If they choose “Twice A Day”, the two Reminder values will default to “1:00 PM” and “7:00 pm” respectively. If they choose “Once A Day”, the one Reminder value will default to “7:00 pm”. For additional information about the care team defined configuration variables, please refer to the Configuration Variables page.

The Steps node has several global configuration values that are preset within the care plan template that cannot be viewed or modified by the care team. They consist of the following: 1) Ramp Up Interval; 2) Ramp Up Start Week Number; 3) Ramp Up Number Week Frequency; 4) Percent Required Mid-Day.

Ramp Up Interval is the number of steps that the patient's daily steps goal for the upcoming week will be increased by when they hit the daily steps goal for the required number of days during the previous week. The value is defined in number of steps at “400”. Ramp Up Start Week Number is the week number in the care plan for which the patient will start having their daily steps goal increased when they hit the daily steps goal for the required number of days during the previous week. The default value for this parameter is “3”. Ramp Up Number Week Frequency is the frequency for which the patient's daily steps goal will possibly be increased. The value is defined in number of weeks at “1”. Percent Required Mid-Day is the percentage of steps that the patient must have not yet achieved by the configured first Reminder to receive an encouragement message. The default value for this parameter is “50”. Percent Daily Target Met is the threshold, over a weekly timeframe, to send a kudos message for the weekly notification. The patient must have met at least 71% of the daily steps goals for the week (which equates to 5 of 7 days). The default value for this parameter is “71”.

FIG. 8b is a flow chart illustration of the Care Plan Process protocol of the Activity-Steps component of a the patient's personal care plan. Once a patient has activated their personalized care on their mobile device, their care plan duration clock starts. To begin tracking their activity, they simply have to put on their step monitoring device and walk each day to have their measurements captured. Week 1 and Week 2 of the care plan will have a daily steps goal that depends on the Activity Level selected. Every week, starting in Week 3, and continuing each week of the care plan, if the patient has averaged a positive number of steps greater than or equal to 50 over their daily goal for the previous week, they will be presented with the option to commit to a daily steps goal increase for the upcoming week. Alternatively, the patient may be presented with the option to commit to a daily steps goal decrease for the upcoming week if they have averaged 50 steps below their daily goal for the previous week. The patient will receive the notification on Monday mornings at 9:00 am local time informing them of their progress, and a proposed new daily steps goal moving forward. If the patient accepts and commits to the new daily steps goal, their care plan will be updated accordingly. If the patient ignores or declines the new daily steps goal, their care plan will not be updated.

FIG. 8c is a flow chart illustration of the Weekly Process protocol of the Activity-Steps component of a the patient's personal care plan. The weekly timeframe for the Activity-Steps component will be set to start on Monday morning at 12:00 am and end on Sunday night at 11:59 pm for all patients.

FIG. 8d is a flow chart illustration of the Daily Process protocol of the Activity-Steps component of a the patient's personal care plan. The daily timeframe for the Steps node of the Activity component will be set to start early in the morning at 12:00 am and end late at night at 11:59 pm for all patients. Upon achieving their daily steps goal anytime throughout the day, the patient will enter the Daily Goal Met Protocol. If the patient has not achieved their daily steps goal by the configured first Reminder, the patient will enter the First Reminder Protocol. If the patient has not achieved their daily steps goal by the configured second Reminder, the patient will enter the Second Reminder Protocol.

FIG. 8e is a flow chart illustration of the First Reminder protocol of the Activity-Steps component of a the patient's personal care plan. If a patient has not achieved their daily steps goal by the configured first Reminder, they will enter this protocol. The Percent Required Mid-Day global configuration value, multiplied by the daily steps goal for the day is what drives this protocol. If the patient has not met the number of steps, the patient will receive an encouragement notification. Otherwise, the protocol will end.

FIG. 8f is a flow chart illustration of the Second Reminder protocol of the Activity-Steps component of a the patient's personal care plan. If the patient has not achieved their Steps goal by the configured second Reminder, they will enter this protocol for the encouragement or troubleshooting workflows depending on if any steps or some steps were captured. The patient's daily goal for the day is what drives this protocol.

FIG. 8g is a flow chart illustration of the Daily Goal Met protocol of the Activity-Steps component of a the patient's personal care plan. If the patient achieves their daily steps goal at any point in the day, the Daily Goal Protocol will be initiated. The patient's daily steps goal is what drives this protocol. If the patient has met the number of steps, the patient will receive a kudos notification. Otherwise, the protocol will end.

As part of the Nutrition component within the Type II Diabetes Care Plan, there is a Journal node that can be configured and implemented for each patient. The Journal node is used to monitor consumption behaviors of a patient. Information about a patient's diet, even in its most basic form, can be invaluable to the care team when evaluating patients. The Nutrition-Journal node for a patient is optional as a care team member may only require its use by the patient under certain circumstances. The care team may add up to 20 journals per patient throughout the duration of the care plan in the Journal node. However, for any given date, only one journal may be active at a time. There are the following configuration fields: 1) Description; 2) Frequency; 3) Reminder; 4) Start Date; and 5) End Date

“Description” is a required text field that allows the care team to define name for the configured journal. The name selected does not have to be unique as it only serves as a reference for the care team. “Frequency” is a pick list that allows the care team member to define the number of times per day that the patient must provide their consumption behaviors. The options available are “Once A Day”, “Twice A Day”, and “Three Times A Day”. The default value is “Three Times A Day”. Upon setting the “Frequency,” the care team member will be able to set the “Reminder” time(s). If they choose “Three Times A Day”, the three reminder values will default to “9:00 am”, “1:00 pm”, and “7:00 pm”. If they choose “Twice A Day”, the two reminder values will default to “1:00 pm”, and “7:00 pm”. If they choose, “Once A Day”, the reminder value will default to “7:00 pm”. The list options for Reminder are in 30-minute increments spanning the 24 hours of a day. For example, the options are: 12:00 am, 12:30 am, . . . , 11:00 pm, 11:30 pm. “Start Date” and “End Date” are date fields with calendar helpers allow the care team member to define the start and end dates for the given journal. For “Start Date,” instead of defining a date, the care team member can simply select the Immediately checkbox, which will default the current date. For “End Date,” instead of defining a date, the care team member can simply select the None checkbox, which will keep logging active throughout the care plan duration.

The Nutrition-Journal node has several global configuration values that are preset within the care plan template that cannot be viewed or modified by the care team. They consist of the following: 1) Max Scheduled Missed Journal; 2) Reminder Interval Missed Journal; 3) Max Scheduled Missed Journal is the number of times that follow up reminders are sent after not providing consumption behaviors. The value is defined as “1”. Reminder Interval is the interval of time in minutes between the initial reminder and follow up reminders sent after not providing consumption behaviors. The value is defined as “15”.

FIG. 9b is a flow chart illustration of the Care Plan Process protocol of the Nutrition-Journal component of a the patient's personal care plan. Once a patient has activated their personalized care plan on their mobile device, their care plan duration clock starts. If a configured journal was not defined during the care plan configuration, the ability to provide consumption behavior is still available in the mobile app, but is voluntary with no scheduled reminders. If a journal was defined during the care plan configuration or at any point during the care plan duration, the patient will be expected to provide their consumption behaviors as long as it is active, which means that “today” is a date after the Start Date and prior to the End Date.

The weekly timeframe for the Journal node will be set to start on Monday morning at 12:00 am and end on Sunday night at 11:59 pm for all patients. The tracking of consumption behaviors is defined within the Scheduled Journal Process. The daily timeframe for the Journal node will be set to start early in the morning at 12:00 am and end late at night at 11:59 pm for all patients. The tracking of consumption behaviors is defined within the Scheduled Journal Process. For a configured scheduled journal, the patient will be able to self-report what they have consumed. If the patient does not, the patient will receive a reminder per the scheduled Frequency and Reminder asking them to provide what they have consumed. If the patient does not reply to the reminders, this will trigger follow up reminders to be sent per the global configuration values. After 48 hours, which will always include today and yesterday, the patient cannot update their response for a logged consumption behavior. Upon providing the most recent consumption behavior, the patient will be able to provide a text description, a whole value for estimated servings, and a picture of what was consumed. In the Care Team Portal, if in need of contextual information when evaluating other metrics such as weight or glucose, the care team can review the patient's journal.

While the foregoing is directed to embodiments of the present disclosure, other and further embodiments of the disclosure may be devised without departing from the basic scope thereof, and the scope thereof is determined by the claims that follow.

We claim:

1. A method for using a care plan assigned to a patient to manage a chronic illness for said patient in response to data observed from the patient, the method comprising:
receiving data corresponding to one or more types of biometric data for the patient, wherein the care plan includes one or more biometric thresholds corresponding to each of the one or more types of biometric data;
receiving one or more reported symptoms for the patient, wherein the care plan includes one or more conditions corresponding to each of the one or more reported symptoms; and
upon determining that at least one of (i) the received data satisfy at least one of the biometric thresholds and (ii) the one or more reported symptoms satisfy at least one of the one or more conditions, identifying an event rule for providing a nonadherence notification to said patient and further providing a suggested action by said patient to bring said patient into adherence with said care plan.

2. The method of claim 1, wherein the one or more types of biometric data includes at least one of a blood sugar measurement, a weight measure, or an activity measurement of a patient.

3. The method of claim 1, wherein the event rule specifies one or more tasks for the patient to perform to bring the patient into adherence with specified parameters of said care plan.

4. The method of claim 3, further comprising: upon determining that subsequently observed biometric data satisfies at least one of said thresholds or subsequently reported symptoms satisfy one or more second conditions, identifying a second event rule corresponding to the satisfied second conditions.

5. The method of claim 4, wherein the second event rule comprises an instruction for the patient to contact a care provider.

6. The method of claim 5, further comprising, receiving an instruction from said care provider notifying the patient to perform a second task to bring said patient into adherence with said care plan.

7. The method of claim 6, further comprising: upon determining that subsequently observed biometric data satisfies at least one of said thresholds or subsequently reported symptoms satisfies one or more of the conditions, identifying a third event rule corresponding to the satisfied second conditions.

8. A computer-readable storage medium storing instructions, which, when executed on a processor, performs an operation for using a care plan assigned to a patient to manage a chronic illness for said patient in response to data observed from the patient, the operation comprising: receiving data corresponding to one or more types of biometric data for the patient, wherein the care plan includes one or more biometric thresholds corresponding to each of the one or more types of biometric data; receiving one or more reported symptoms for the patient, wherein the care plan includes one or more conditions corresponding to the one or more reported symptoms; and upon determining that at least one of (i) the received data satisfy at least one of the biometric thresholds and (ii) the one or more reported symptoms satisfy at least one of the one or more conditions, identifying an event rule for providing a nonadherence notification to said patient and further providing a suggested action by said patient to bring said patient into adherence with said care plan.

9. The computer readable storage medium of claim 8, wherein the one or more types of biometric data includes at least one of a blood sugar measure, a weight measure, or an activity measure of a patient.

10. The computer-readable storage medium of claim 8, wherein the event rule specifies one or more tasks for the patient to perform to bring the patient into adherence with specified parameters of said care plan.

11. The computer-readable storage medium of claim 10, wherein the operation further comprises: upon determining that subsequently observed biometric data satisfies at least one of said thresholds or subsequently reported symptoms satisfy one or more of the conditions, identifying a second event rule corresponding to the satisfied second conditions.

12. The computer-readable storage medium of claim 11, wherein the second event rule comprises an instruction for the patient to contact a care provider.

13. The computer readable storage medium of claim 12, further comprising, receiving an instruction from said care provider notifying the patient to perform a second task to bring said patient into adherence with said care plan.

14. The computer readable storage medium of claim 13, further comprising: upon determining that subsequently observed biometric data satisfies at least one of said thresholds or subsequently reported symptoms satisfies one or more of the conditions, identifying a third event rule corresponding to the satisfied second conditions.

15. A system, comprising: a processor, and a memory storing one or more application programs configured to perform an operation for using a care plan assigned to a patient to manage a chronic illness for said patient in response to data observed from the patient, the operation comprising: receiving data corresponding to one or more types of biometric data for the patient, wherein the care plan includes one or more biometric thresholds corresponding to each of the one or more types of biometric data, receiving one or more reported symptoms for the patient, wherein the care plan includes one or more conditions corresponding to the one or more reported symptoms, and upon determining that at least one of (i) the received data satisfy at least one of the biometric thresholds and (ii) the one or more reported symptoms satisfy at least one of the one or more conditions, identifying an event rule for providing a nonadherence notification to said patient and further providing a suggested action by said patient to bring said patient into adherence with said care plan.

16. The system of claim 15, wherein the one or more types of biometric data includes at least one of a blood sugar measure, a weight measure, or an activity measure of a patient.

17. The system of claim 15, wherein the event rule specifies one or more tasks for the patient to perform to bring the patient into adherence with specified parameters of said care plan.

18. The system of claim 17, wherein the operation further comprises: upon determining that subsequently observed biometric data satisfies at least one of said thresholds or subsequently reported symptoms satisfy one or more of the conditions, identifying a second event rule corresponding to the satisfied second conditions.

19. The system of claim 18, wherein the second event rule comprises an instruction for the patient to contact a care provider.

20. The system of claim 19, wherein the operation further comprises evaluating past treatment history of the patient; and identifying a third task for the patient to perform based on the past treatment history.
